(19) United States
(12) Patent Application Publication
(10) Pub. No.: US 2024/0041386 A1
(43) Pub. Date: Feb. 8, 2024

(54) MICROCHIP BASED RECORDING OF NEUROVASCULAR ACTIVITY AND BEHAVIOR

(71) Applicant: NEUREFLECT, INC., Santa Monica, CA (US)

(72) Inventors: Andrew CHARLES, Mammoth Lakes, CA (US); Guido FAAS, Santa Monica, CA (US); Dmitri YOUSEF YENGEJ, Los Angeles, CA (US)

(73) Assignee: NEUREFLECT, INC., Santa Monica, CA (US)

(21) Appl. No.: 18/489,801

(22) Filed: Oct. 18, 2023

Related U.S. Application Data
(63) Continuation of application No. PCT/US2022/025397, filed on Apr. 19, 2022.
(60) Provisional application No. 63/176,832, filed on Apr. 19, 2021.

(51) Int. Cl. A61B 5/00; A61B 5/11; A61B 5/02

(52) U.S. Cl. CPC A61B 5/4064; A61B 5/0002; A61B 5/1126; A61B 5/0071; A61B 5/02007; A61B 5/6814; A61B 2503/40

(57) ABSTRACT

A minimally invasive, microchip-based system which enables continuous long-term recording of subjects (e.g., freely moving rodents) under study including subject behavior, subject brain neurovascular activity, subject heart rate, subject intercranial pressure, subject temperature, and subject head movement is described herein. These systems may also include microchips configured to deliver stimuli such as heat stimuli and measure these various parameters of subjects before, during, and after stimuli. The systems may afford measurement protocols in one or more subjects such as conditioned place/aversion assays and/or pharmacological or medical device testing.

### CROSS REFERENCE TO RELATED APPLICATIONS

The present application claims priority to U.S. App. No. 63/176,832, filed Apr. 19, 2021, which is hereby incorporated by reference in its entirety.

### FIELD OF DISCLOSURE

The present disclosure is related to systems and method for the measurement of neurovascular activity and behavior in a subject. This disclosure relates to continuous measurement protocols that may occur over longer periods of time (e.g., five or more or ten or more or twenty or more or twenty-five or more sleep-wake cycles) and that are minimally invasive to the subject (e.g., unanesthetized subjects).

### BACKGROUND

Long term monitoring of brain activity and behavior in rodent models can provide important insights into basic mechanisms of normal brain function, alterations in function that result from neurological or systemic diseases, and potential treatments for these diseases. A challenge in the investigation of brain activity in the laboratory has been the invasiveness of the procedures required for data collection. Triggering and recording brain activity in animal models typically requires breaching the skull, and the majority of studies have been performed under anesthesia. Studies performed in awake animals may require head fixation and are often limited in duration because of the invasiveness of the techniques and the size of the recording equipment.

Electrophysiological recordings including electroencephalography (EEG) are one method for continuous recording of brain activity which can be performed with wired or wireless configurations. This approach typically requires invasive implantation of recording electrodes.

An alternative strategy is to use optical approaches. Genetically encoded or virally expressed fluorescent indicators enable investigation of brain cellular activity in vivo. While this approach enables recording with exceptionally high spatial resolution, previous used methods using this approach employed head-mounted optics that require breaching of the skull. Also, the size and weight of the previously used optics may require head fixation, limiting the movement of the animal and the duration of recording.

An alternative strategy is the measurement of reflected light from brain tissue (optical intrinsic signal or OIS). This method has been used as a technique for measuring brain neurovascular activity (Kim et al., Sci Rep 7 (2017): 13148; Kura et al., J Neural Eng 15 (2018): 035003; Friedman et al., Neuroimage 221 (2020): 117188, each of which are incorporated by reference in their entirety and particularly in relation to OIS protocols and measurements). Briefly, OIS is comprised of multiple components, including blood, cell volume/extracellular space, and intracellular organelles such as mitochondria. The relative contribution of these components varies with the wavelength of light that is reflected. For example, with green light illumination, the primary component of OIS is tissue blood volume which, under conditions of normal neurovascular coupling, is an indicator of neural activity.

While OIS techniques do not typically have the capacity for high spatial resolution as compared with fluorescence imaging techniques, measurements can be performed less invasively with simpler optical components, and do not require a fluorescent indicator. Previous OIS recording in vivo has been performed primarily through cranial windows, with traditional microscopy in anesthetized mice, or with head-mounted optics in mice in vivo as disclosed in Senarathna et al., Nat Commun 10 (2019): 99, which is hereby incorporated by reference in its entirety and particularly in relation to OIS protocols and measurements.

Optical approaches can also be used to trigger brain activity in mice expressing channels or receptors that are activated or inhibited by light. This method has been highly useful in probing the role of specific cell types in the nervous system but requires access of light stimulation to the target tissue, which for in vivo stimulation has typically been performed using implanted fiber optics.

Previous studies have used OIS recording and optical stimulation to trigger and record brain activity in mice, specifically cortical spreading depression (CSD), the slowly propagated wave of brain activity that is believed to be the physiological substrate of the migraine aura, in awake and freely moving animals with a head-fixed fiber optic system (Houben et al., J Cereb Blood Flow Metab 37 (2017): 1641-1655, which is which is hereby incorporated by reference in its entirety and particularly in relation to OIS protocols and measurements involving CSD). These studies were limited, however, by the invasive nature of the recording and the weight of the head-mounted equipment.

These measurements are invasive to subjects, require limitations on subject mobility (i.e., subjects are not “free moving” in an enclosure), require anesthetization of the subject, do not measure neural activity over prolonged periods or with sufficient frequency to elucidate the complex neural activity associated with neural conditions. It is therefore an object of this disclosure to provide apparatuses, systems, and methods which overcome these problems with previous measurement protocols.

### SUMMARY

In accordance with the foregoing objectives and others, the present disclosure provides microchips (or substrates comprising the requisite components), microelectronics, systems, and methods for providing enhanced measurement capabilities of neural activity. The present disclosure may utilize a single integrated microchip system and/or microelectronics system that, when attached to an intact skull, is capable of transcranial recording of brain neurovascular activity as indicated by spectral measurements such as OIS or fluorescence measurements, measurement of multi-directional head movement with a highly sensitive sensor, optical triggering, or inhibition of brain activity (particularly in mice expressing opsins like channelrhodopsin or halorhodopsin), or combinations thereof. Additional components of the system include sensors for temperature and intracranial pressure, and microelectronics for delivery of a calibrated heat stimulus. Each of these components may be independently placed on one or more microchip-based systems configured to be affixed to the skull of a subject. The signals to and from each microchip may be independently transmitted from a microchip system enabling microchip-based recording of neurovascular activity and behavior in a subject. In some embodiments, a single microchip comprises each indicated component. In some embodiments, each indicated microelectronics component (or groups thereof) are supplied separate microchips (e.g., a substrate having an integrated circuit for accommodating the component) which together form a microchip/microelectronics system for simultaneous measurement and stimulation.

The present disclosure also includes systems which include one or more of these microchips. For example, when the systems involve an enclosure for the subjects, a camera-based tracking system may enable real-time localization. These systems may allow for conditioned place preference/aversion assays. For example, in a system with real-time localization, a subject may be conditioned by providing an optical or heat stimulus only when the subject is in a specific area of the enclosure. Measurements such as spectral measurements (e.g., OIS, fluorescence), intracranial pressure, head movements, skull temperature, or combinations thereof may be taken with one or more of the microchip systems of the present disclosure before, during, and/or after conditioning of the subject.

The apparatuses, systems, and methods of the present disclosure have been used to characterize brain blood flow and behavior throughout the sleep-wake cycle, to intermittently trigger and record CSD, to monitor intracranial pressure, to measure behavioral responses to a calibrated heat stimulus, heart rate, and to track temperature changes associated with the estrous cycle over periods of up to 60 days.

The microchips, microelectronics and systems of the present disclosure may enable continuous recording and triggering of neurovascular activity, heart rate, and behavior in freely behaving rodents over weeks. These may be used to characterize physiological and behavioral changes associated with the sleep/wake cycle over extended time periods. In particular, these systems may be used with freely behaving mice expressing channelrhodopsin to trigger and record cortical spreading depression (CSD). The microchips, microelectronics, systems, and methods of the present disclosure offer the capability for continuous measurements of subjects such as the measurement of neurovascular responses to CSD in anesthetized, unanesthetized, awake, sleeping subjects and illustrate that these states are remarkably different.

Microchip systems of the present disclosure are typically used for the detection of neurovascular activity in a subject (e.g., mouse, rat, primate, human, dog). The microchip systems may be attachable to the skull (e.g., reversibly attachable to the skull such as with adhesives including super glue or acrylic based adhesives such as cyanoacrylate). Typically, the adhesive does not interfere with the detection of neurovascular activity (e.g., no alteration in a measured parameter or behavior is observed following adhesive attachment). The microchip system may be dimensioned such that it comprises a perimeter (e.g., a raised edge) to allow for the microchip system to be adhered to the skull.

The microchip system may comprise:
a) a source of electromagnetic measurement radiation (or a first source of electromagnetic radiation) arranged on said microchip system such that when said microchip system is attached to said skull, at least a portion of said electromagnetic radiation from said source penetrates said skull and is reflected from or elicits fluorescence from underlying neural tissue;
b) an optical sensor arranged on said microchip system such that when said microchip system is attached to said skull, said optical sensor is configured to sense a portion of said reflected electromagnetic radiation or fluorescence from neural tissue underlying said skull;
wherein changes in the reflected electromagnetic radiation or fluorescence correlate with said neurovascular activity; and
wherein said microchip system is configured to receive signal from said optical sensor and said microchip system is configured to transmit an electrical signal corresponding to at least one property of said electromagnetic radiation. In some embodiments, the electromagnetic radiation comprises green light and/or infrared light. For example, the source of electromagnetic measurement radiation may comprise a light emitting diode (or “measurement light emitting diode”). The light emitting diode may emit a spectrum of electromagnetic radiation having one or more λₘₐₓ independently selected from those useful for OIS and/or fluorescence measurements. For example, the light emitting diode may emit a spectrum of electromagnetic radiation having one or more λₘₐₓ independently selected from 275-950 nm (e.g., 275-400 nm, 400-500 nm, 500-650 nm, 650-800 nm, 800-950 nm).

The microchip system may comprise a source of electromagnetic stimulus radiation (or second source of electromagnetic radiation) which may be used to illicit a response in the subject. For example, the microchip system may further comprise a source of electromagnetic stimulus radiation; wherein the electromagnetic stimulus radiation is electromagnetic radiation emitted from said source towards said skull; and wherein said stimulus electromagnetic radiation induces a response in said subject. In some embodiments, the electromagnetic stimulus radiation may be used to induce cortical spreading depression (CSD) in a subject by transmitting light through the skull of subjects which would have such a response. For example, the subject may be a subject expressing light-activated proteins such as channelrhodopsin or halorhodopsin. In various implementations, the source of electromagnetic stimulus radiation comprises a light emitting diode (or “stimulus light emitting diode”).

The microchip system may further comprise one or more (e.g., one, two, three, four, five, six, seven, eight) motion sensors. The motion sensors may comprise one or more accelerometers. The motion sensors may be configured to measure movement and orientation of the head in a particular direction and/or acceleration of the head. The motion sensors may transmit data in order to identify motion in all three directions of a reference frame and/or acceleration (e.g., aX, aY, and aZ) and/or rotation in all three directions (i.e. measurement of the pitch, yaw, and roll of the subject can each be independently measured in one or more motion sensors, e.g. ωX, ωY, ωZ). The reference frame for acceleration and position may be the same or different. The microchip system may further comprise additional sensors to allow for additional and potentially concurrent measurements with the spectral measurements and/or motion/acceleration measurements. For example, the microchip system may comprise a thermal sensor (e.g., thermometer, thermocouple) for measurement of skull surface temperature, a pressure sensor (e.g., piezoresistive pressure sensors) for measurement of intracranial pressure.

Additionally, the microchip system may further comprise one or more components to stimulate the subject (in a manner different than with the electromagnetic stimulus radiation directed through the skull) and consequently measure reactions to the stimulus with the other sensors described herein. For example, in some embodiments, the microchip system may comprise a heat source (e.g. a resistive heater). The heat source may be used to measure heat-pain thresholds in concert with the other measurements performed, or to deliver a calibrated painful stimulus. The calibrated heat stimulus may be calibrated such that the temperature and timing (duration) of the thermal stimulus can be controlled. The heat source may be configured to deliver to the subject a pain stimulus by applying a specified temperature (e.g., a temperature from 50° C.-200° C.) for a specified duration (e.g., from 1 s-1 hr) around a “calibrated pain threshold.” The calibrated pain threshold may be identified on an individual subject basis, or through averaged measurements over many subjects. In some embodiments, the microchip systems of the present disclosure measure one or more parameters about the subject while the pain stimulus is being applied. In some embodiments, the pain stimulus is used in a conditioned place aversion type protocol, wherein when the subject is identified as being in a particular location in an enclosure (e.g., with video tracking), the calibrated pain stimulus is applied.

Also provided are microchips and/or microelectronics systems for the detection of behavior in a subject comprising a sensor selected from a motion sensor, a thermal sensor for measurement of the skull surface temperature, or a pressure sensor for the measurement of intracranial pressure;
wherein the microchip is configured to be affixed to the skull of the subject;
measurements from the sensor are correlated with behavior of the subject; and the microchip is configured to transmit an electrical signal from the sensor corresponding to the measurement.

The present disclosure also includes microchip systems for the detection of neurovascular activity and/or behavior of a subject. These microchip systems may comprise two or more microchips configured to be attached to the skull of the subject; the two or more microchips selected from a spectral measurement microchip, a behavior measurement microchip, and a stimulus microchip:
wherein the spectral measurement microchip system comprises:
   a) a source of electromagnetic measurement radiation arranged on the microchip system such that when the spectral measurement microchip system is attached to the skull, at least a portion of the electromagnetic radiation from the source reflects off of neural tissue and/or triggers fluorescence from neural tissue comprising one or more fluorescent molecules underlying the skull; and
   b) an optical sensor arranged on the microchip system such that when the microchip system is attached to the skull, the optical sensor is configured to sense a portion of the reflected electromagnetic radiation off of or fluorescence from neural tissue comprising one or more fluorescent molecules underlying the skull;
wherein changes in the reflected and/or fluoresced electromagnetic radiation correlate with the neurovascular activity; and
wherein the spectral measurement microchip system is configured to receive signal from the optical sensor and the microchip system is configured to transmit an electrical signal corresponding to at least one property of the electromagnetic radiation;
said behavioral measurement microchip system comprises:
   a) a sensor selected from a motion sensor, a thermal sensor for measurement of the skull surface temperature, or a pressure sensor for the measurement of intracranial pressure;
measurements from the sensor are correlated with behavior of the subject; and the behavioral measurement microchip system is configured to transmit an electrical signal from the sensor corresponding to the measurement; and
said stimulus microchip system comprises a stimulus configured to provide a stimulus to the subject (e.g., a spectral stimulus from a source of electromagnetic stimulus radiation, a thermal stimulus). In some embodiments, the microchip system and/or microelectronics system comprises a spectral measurement microchip system and a behavior measurement microchip system. In some embodiments, the microchip system comprises two or more behavioral measurement microchips. In some embodiments, the microchip system and/or microelectronics system comprises a spectral measurement microchip a behavior measurement microchip, and a stimulus microchip. In some embodiments, the microchip system comprises a spectral measurement microchip, a microchip configured to measure intracranial pressure, a microchip configured to measure skull surface temperature, and a microchip comprising a motion sensor. In various implementations, the stimulus microchip system is configured to deliver a heat stimulus to the subject. In some embodiments, the stimulus microchip system comprises a source of electromagnetic stimulus radiation, wherein radiation emitted from the source towards neural tissue underlying the skull; and
the stimulus electromagnetic radiation induces a response in the subject. These microchips and/or microelectronics systems may be connected on a flexible printed circuit board.

The microchip system may have a chip architecture configured to allow transmission of the sensor data to a recording device configured to record this data. The recording device may be, for example, a computer, microcontroller, solid state memory storage. In some embodiments, the chip architecture is also configured to transmit, from an external source such as microcontroller, appropriate signals to the components of the chip (e.g., the source of electromagnetic measurement radiation, the source of electromagnetic stimulus radiation, the heat stimulus, the optical sensor, the pressure sensor, the motion sensor) to allow their independent operation. In various implementations, the microchip is controlled via a server configured to interface with a microcontroller and the microcontroller is configured to interface with the microchip. In some implementations, the microcontroller is configured to automatically deliver a stimulus (e.g., triggering a CSD or delivering a heat stimulus) in response to information obtained from one or more of the sensors (e.g., during the sleep or wake state as determined by the motion sensor, or when the subject is in a specific part of the enclosure as determined by a camera sending location measurements to the microcontroller). In some embodiments, the microchip system comprises a connector for a wired connection between said microchip system and said recording device. In various implementations, the microchip system further comprises a battery connected to the source of electromagnetic measurement radiation and/or the source of electromagnetic stimulus radiation and/or the optical sensor and/or the motion sensor and/or the thermal sensor and/or the pressure sensor and/or the heat source. In some embodiments, the microchip system comprises radio-frequency identification components.

The systems for the detection of neurovascular activity in a subject may comprise:
a) a microchip configured to measure optical signal (e.g., OIS, fluorescence signal) while being affixed to the skill of a subject, and
b) a recording system in communication with the microchip, wherein each microchip transmits optionally simultaneous measurements from each sensor independently (e.g., measurements from the optical sensor, measurements, from the motion sensor, measurements from the thermal sensor, measurements from the pressure sensor, combinations thereof) to the recording system.

The microchip system may comprise:
a) a source of electromagnetic measurement radiation (or a first source of electromagnetic radiation) arranged on said microchip system such that when said microchip system is attached to said skull, at least a portion of said electromagnetic radiation from said source reflects off of neural tissue and/or is absorbed by a fluorescent molecule (e.g., fluorescent molecules in the neural tissue of the subject) underlying said skull;
b) an optical sensor arranged on said microchip system such that when said microchip system is attached to said skull, said optical sensor is configured to senses a portion of said reflected electromagnetic radiation from neural tissue or fluorescent molecules underlying said skull;
wherein changes in the reflected electromagnetic radiation or fluorescence correlate with said neurovascular activity; and
wherein said microchip system is configured to receive signal from said optical sensor and said microchip system is configured to transmit an electrical signal corresponding to at least one property of said electromagnetic radiation and/or fluorescence. In some embodiments, the recording system is physically linked (e.g., through a wire) to the microchip system. In various implementations, the recording system comprises a processor or a microcontroller which interfaces with said microchip system; and the processor or microcontroller can receive measurement data from each sensor on the microchip system and/or control components of the microchip system (e.g., the source of electromagnetic measurement radiation and/or the source of electromagnetic stimulus radiation and/or the optical sensor and/or the motion sensor and/or the thermal sensor and/or the pressure sensor and/or the heat source). In some embodiments, the recording system comprises a microcontroller in communication with a server which may simultaneously collect data from multiple subjects.

In certain implementations, the system further comprises one or more cameras for monitoring the movement of said subject (e.g., a subject having one or more microchips affixed to the skull) in said enclosure. In some embodiments, the enclosure comprises a flooring material offering a contrast in color with the subject such that the camera may continuously monitor subject position in the enclosure (and, correlate that position, for example, with the other data being measured such as the data from the optical sensor, the data from the motion sensor, the data from the pressure sensor, the data from the thermal sensor, or combinations thereof). For example, the system may comprise a microcontroller and optionally a camera or cameras, the microchip comprises a motion sensor, and the microcontroller is configured to identify awake and sleep cycles of said subject based on measurements transmitted thereto from said motion sensor and/or said camera. In some embodiments, information about the subject, such as the position in the enclosure or cycle (or place in the sleep/awake cycle) may be used to determine whether to transmit one or more signals back to the microchip system (e.g., from the microcontroller, from the server, from a processor). In various implementations, the cycle may be determined by the microcontroller, and the microcontroller may automatically send a stimulus to any microchip affixed to the skull of the subject, such that an optical or heat stimulus can be delivered automatically in response to data from one or more sensors and/or the camera, such as specific sleep/wake state or a specific location within the enclosure.

Methods for the detection of neurovascular activity in a subject having a microchip configured to measure optical signal (e.g., OIS, fluorescence signal) while being affixed to the skill of a subject are also provided, comprising:
a) emitting electromagnetic measurement radiation from a source of electromagnetic measurement radiation on the microchip system to reflect off of or elicit fluorescence from the neural tissue underlying the skull or fluorescent molecules therein of the subject; and
b) measuring the reflected electromagnetic measurement radiation and/or fluorescence with an optical sensor on the microchip;
wherein changes in the reflected electromagnetic radiation and/or fluorescence correlate with said neurovascular activity.

The method for the detection of neurovascular activity in one or more subjects may comprise:
a) placing each of said one or more subjects into an enclosure in one of the systems according; wherein each of said one or more subjects has said microchip affixed to the skull of said subject for independent measurement of optical (e.g., OIS, fluoresence) signal;
b) emitting electromagnetic radiation from sources of electromagnetic measurement radiation on the microchip to reflect off of and/or elicit fluorescence from the neural tissue or fluorescent molecules therein underlying the skull of each of said subject; and
c) measuring the reflected light and/or fluorescence in an optical sensor from each of said microchips;
wherein changes in the reflected electromagnetic radiation and/or fluorescence correlate with said neurovascular activity. In some embodiments, the method may comprise the simultaneous detection of from one to 100 or one to thirty or one to ten or one to five subjects, where each microchip transmits sensor data to a server (e.g., via microcontrollers in each individual system). In various embodiments, the server aggregates the data collected from each individual microchip. In various implementations, the emitting and measuring steps occur continuously (by which is meant that signals are sent to the recording device as limited by the sensors and/or transmission frequency in the microchip system) occur over more than 30 sleep-wake cycles of the subjects. In some embodiments, the one or more subjects are monitored continuously with a signal frequency sent to the recording device from each sensor independently selected from frequencies less than (or from 0.1 Hz to) 1 kHz. In some embodiments the method comprises recording and stimulation in rodent models of neurological or systemic disease. In some embodiments, the method further comprises administering to said subject a candidate substance to evaluate said candidate substance for effects on brain neurovascular activity and behavior, and on different features of the sleep-wake cycle including REM sleep.

In particular embodiments, a method of monitoring cortical spreading depression (CSD) in a subject (e.g., rat, mouse) expressing channelrhodopsin is provided, wherein said subject expressing channelrhodopsin has a microchip system attached to the skull of said subject, and said microchip system comprises:
a) a source of electromagnetic measurement radiation arranged on said microchip system such that when said microchip system is attached to said skull, at least a portion of said electromagnetic radiation from said source reflects off of neural tissue underlying said skull;
b) an optical sensor arranged on said microchip system such that when said microchip system is attached to said skull, said optical sensor is configured to sense a portion of said reflected electromagnetic radiation off of neural tissue underlying said skull; and
c) a source of electromagnetic stimulus radiation; wherein said electromagnetic stimulus radiation is electromagnetic radiation emitted from said source towards neural tissue underlying said skull; and
wherein the stimulus electromagnetic radiation induces CSD in said subject.
wherein changes in the reflected electromagnetic measurement radiation correlate with the neurovascular activity of the subject; and
wherein the microchip is configured to receive signal from said optical sensor and said microchip is configured to transmit an electrical signal corresponding to at least one property of said electromagnetic radiation;
the method comprising:
a) emitting electromagnetic stimulus radiation from said stimulus radiation source to induce CSD in said subject;
b) emitting electromagnetic measurement radiation from said source of electromagnetic measurement radiation; and
c) measuring the reflected electromagnetic measurement radiation with said optical sensor.

In some embodiments, the method further comprises administering to said subject a candidate substance to evaluate said candidate substance for the treatment of said CSD or a disease, disorder, or condition associated therewith (e.g., migraine aura, brain ischemia, seizures). The emitting and measuring steps may, for example, occur over more than 5 (e.g., more than 10, more than 20, more than 40, more than 60, from 5 to 100, from 50 to 100) sleep-wake cycles of said subject.

Methods for the measurement of the heart rate in a subject are also provided. These methods may comprise measuring the OIS for a period of time to identify the oscillation frequency in the OIS appearing within an appropriate heart rate range for the subject. For example, the appropriate heart rate range for a rodent may be from 300-1200 bpm (e.g., from 400 to 800 bpm). In some embodiments, the methods involve the use of the microchip systems of the present disclosure. For example, the subject may have a microchip system attached to the skull, and the microchip system comprises an optical microchip system having:
a) a source of electromagnetic measurement radiation arranged on said microchip system such that when said microchip system is attached to said skull, at least a portion of said electromagnetic radiation from said source reflects off of or fluoresce from neural tissue underlying the skull of each of said subject;
b) an optical sensor arranged on said microchip system such that when said microchip system is attached to said skull, said optical sensor is configured to senses a portion of said reflected electromagnetic radiation and/or fluorescence radiation; and
wherein changes in the reflected electromagnetic measurement radiation correlate with said neurovascular activity; and
wherein said microchip system is configured to receive signal from said optical sensor and said microchip system is configured to transmit an electrical signal corresponding to at least one property of said electromagnetic radiation;
and the detecting comprising:
a) emitting electromagnetic measurement radiation from said source of electromagnetic measurement radiation; and
b) measuring the reflected electromagnetic measurement radiation with said optical sensor to form the OIS over time.

The method may correlate the OIS to a heart rate. For example, the method may comprise performing spectral analysis (e.g., Fourier Transform such as Fast Fourier Transform) to identify the oscillation frequency in the OIS. In some embodiments, the method may comprise characterizing a disease state of the subject based on the OIS measured heart rate.

### BRIEF DESCRIPTION OF FIGURES

FIG. 1 shows a perspective view of one of the flexprint design of the microchip system of the present disclosure including dimensions. The central IOS chip architecture is coupled with an optical sensor (r), green IOS illumination LEDs (g), blue stimulation LED (b), motion sensor (m), and USB connector (c). The bottom side may be glued onto the mouse skull.

FIG. 2 shows three perspective views of a microchip system of the present disclosure having optical sensor (r), green IOS illumination LEDs (g), blue stimulation LED (b), motion sensor (m), and circuitboard flap (flexprint) shaped to mate with a locking FFC/FPC connector (c) connected to the microchip system through flex print connections.

FIG. 3 illustrates an exemplary system of the present disclosure.

FIG. 4 provides perspective views of two different systems with vibration systems of the present disclosure.

FIG. 5 (FIGS. 5A and B) provides exemplary analysis software that may be used with and in the systems and methods of the present disclosure. FIG. 5A provides a general flow chart illustrating potential machine learning and analysis software for one or a group of subjects. FIG. 5B provides a general schematic where the software operates to maintain the wake state of the subject under study.

FIG. 6 (FIGS. 6A-6F) shows the microchip-based approach of the present disclosure which affords minimally invasive triggering and recording brain activity in awake and freely moving animals. FIG. 6A is a schematic of chip system including green light-emitting diode (g) for OIS recording, optical sensor (r) for recording OIS, blue light (b) for stimulation of channelrhodopsin, and motion sensor (m). FIG. 6B illustrates motion sensor recording acceleration in three directions (aX, aY, aZ) as well as angular momentum (ωX, ωY, ωZ). FIG. 6C illustrates is an image of a manufactured version of the chip system with the components described in FIG. 6A. The included connector (c) allows for simple attachment/detachment of the mouse to the recording setup for data transmission. FIG. 6D illustrates how a real-time video tracking system can enable continuous recording of the location of the mouse within the enclosure. The white line represents the last 20 detected locations of the mouse, showing its most recent movement (coordinates illustrated on left of image). FIG. 6E is a schematic overview of the recording setups showing the commutator (COM) which allows free movement of mouse with wired connection between chip and microcontroller. Microcontrollers from multiple enclosures are connected to a central server, which also receives input from other sensors (e.g., room light/dark, ambient temperature, barometric pressure). FIG. 6F is a photograph of two enclosures mounted with a 3D printed superstructure including commutator, camera holder, and holder for microcontroller and 4″ monitor. Room lighting is generated by wall-mounted LED strips that are controlled and monitored by the central server.

FIG. 7 (FIGS. 7A, 7B-I, 7B-II, 7B-III, 7C-I, 7C-II, and 7D) illustrate the validation of chip-based recording and triggering of brain activity in anesthetized mice. In this, and all subsequent figures, OIS is expressed relative to baseline OIS determined as the 24-hour rolling median of the OIS. Downward changes in OIS traces indicate a decrease in CBV and vasoconstriction (increased reflectance) whereas upward changes indicate an increase in CBV and vasodilation. FIG. 7A is a schematic showing reduced reflectance (OIS) of green light when vessels are dilated and there is increased blood volume with increased reflectance when vessels are constricted. FIG. 7B-I illustrates an experimental configuration for recording OIS with a system of microchips (presently disclosed system) and camera (for validation) through window of thinned skull in anesthetized mouse. A light-sensitive microchip (square with asterisk) was glued over a part of the cortex that is affected by the CSD. A glass microelectrode inserted through a small hole in the skull was used to measure the field potential (large black arrow). To measure OIS from the camera image, the average intensity of an area in the field of view is taken (white oval). CSD was triggered by application of 1 M KCl (small white arrow and tube). FIG. 7B-II provides images showing that the cortex readily was visible through the skull in the thinned skull preparation (N). Subsequent images taken during OIS measurements illustrate how CSD is associated with a slowly propagating wave of vasoconstriction and decreased blood volume by contrast-enhanced (taken from 0 s-5 min) images. Asterisk denotes the light-sensitive microchip position; arrows and tube denote KCl application. FIG. 7B-III provides a comparison of traces of OIS showing two (2) CSD events in response to continuous application of KCl. Chip recording (bottom) shows OIS changes parallel to those recorded with camera (middle), as well as field potential recordings (top), validating the chip method of recording of the present disclosure. The OIS camera signal and OIS chip signal slightly deviate from each other because they are measured over different cortical areas thus, will measure slightly different signals. FIG. 7C-I provides images of CSD triggered with light stimulation from an LED (grey dot) and recorded with a microchip (asterisk) in a Thy1Chr mouse. Temporal and spatial characteristics of CSD are similar to those observed with KCl stimulation. FIG. 7C-II is a comparison of OIS traces simultaneously recorded with camera (top) and microchip (bottom) showing similar pattern of change with light-evoked CSD. In this experiment the microchip OIS trace contains an artifact from the blue LED due to the proximity of the optical sensor to the LED. FIG. 7D illustrates how spontaneous bursts of action potentials (top) are correlated with periodic vasodilation as indicated by recording of OIS with microchip (middle). Lower amplitude oscillations within the OIS indicate heart rate and breathing (bottom).

FIG. 8 (FIGS. 8A and 8B) are OIS measurement traces from a microchip of the present disclosure with time resolutions illustrating the high and low frequency oscillations in the OIS trace correlated with heart rate and breathing, respectively. The bottom trace in FIG. 8B is the simultaneous electrophysiological recording in the subject illustrating neurovascular coupling between cerebral blood flow (CBF) as measured by OIS and brain activity.

FIG. 9 (FIGS. 9A-9E) illustrate the continuous recording of cortical blood volume (CBV) and movement in a freely behaving mouse. Representative traces of long term continuous non-invasive microchip recording in a single subject (N=6 total subjects) are shown. FIG. 9A includes measurements taken from microchip measurements over several day/night cycles. The top black trace shows changes in OIS (corresponding with CBV) over a 6-day. Bottom grey trace shows head rotation speed (or angular momentum, ω=(ωX²+ωY²+ωZ²)^{1/2} taken from accelerometers in the microchip) as a measure of overall movement over the same period recorded with the motion sensor chip. Oscillations in CBV and movement are seen to be correlated with the day/night cycle. Central white traces are the rolling average over a 4-hour window, revealing a slow oscillation in average OIS and activity, coinciding with the light cycle. Grey blocks indicate 12-hour dark periods. FIG. 9B shows the changes in OIS and movement over a single day, illustrating a general correlation between CBV and movement during wakefulness and sleep. FIG. 9C is traces from microchip measurement showing changes in OIS (top) and movement (bottom) over a 5-hour period. On the OIS trace, black color indicates wakefulness, whereas grey color (or portion of trace below dashed line) indicates sleep. During sleep, periodic increases in CBV not accompanied by an increase in movement are consistent with periods of REM sleep. A 5-minute rolling median of the angular momentum (black central line in bottom trace) with a threshold (dashed line) was used to automatically detect wakefulness or sleep. FIG. 9D correlates the sleep and awake measurements into a bimodal distribution (left). FIG. 9E shows the OIS recording in a rat with the same periodic increases in CBV as seen in the mice during sleep.

FIG. 10 (FIGS. 10A-10C) illustrates that heart rate analysis possible from the presently disclosed microchip systems and/or microelectronics systems. Representative analysis of heart rate in a single mouse (N=6 total) is shown. FIG. 10A is a trace of head-attached microchip OIS recording showing low-amplitude oscillations that are indicative of the arterial pulse. FIG. 10B provides spectral analysis (of the trace in FIG. 10A) which indicates the frequency of the low-amplitude oscillations consistent with the heart rate. FIG. 10C shows that OIS indicative of CBV (black trace) are generally correlated with heart rate (grey bottom trace).

FIG. 11 illustrates the optical triggering and recording of CSD in a freely behaving mouse using a microchip of the present disclosure. CSD was triggered by blue light stimulation in a Thy1Chr mouse with the microchip. In the top trace, two CSD events that are triggered several days apart are shown, with characteristic decreases in CBV. Expanded trace (bottom) shows typical immediate and sustained decrease in CBV occurring with optically triggered CSD. L denotes onset of blue light stimulation.

FIG. 12 (FIGS. 12A-12C) illustrate different characteristics of CSD in the awake state vs. under anesthesia. FIG. 12A provides traces of mean OIS (N=5, bands represent SEM) with CSD triggered optically (left arrow) in the same animals in the awake state vs. anesthesia. Under anesthesia, the vasoconstriction associated with the CSD was bigger and lasted longer than in the awake state. FIG. 12B provides a comparison of data illustrating the measured differences in overall amplitude and duration of the CSD event, quantified as area under the curve (AUC), from the start of the CSD over a window of 2 h (A: left arrow to right arrow). AUC of the CSD when awake was around 10× smaller AUC than under anesthesia. FIG. 12C provides an additional trace illustrating the differences in IOS signal during CSD during anesthetized sleep as compared to awake.

FIG. 13 provides comparative traces of CSD (stimulation occurring between triangles) as measured by cerebral blood flow (taken from OIS measurements) and the concurrent measurements taken from the motion sensor during asleep (light grey) and awake (dark grey) periods.

FIG. 14 (FIGS. 14A and 14B) include OIS (or “IOS”) traces taken simultaneously with intracranial pressure (ICP) measurements from a microchip system of the present disclosure affixed to the skull of a mouse (FIG. 14A) or a rat (FIG. 14B).

FIG. 15 (FIGS. 15A-15C) relate to measurements performed on microchip systems comprising a heat stimulus. FIG. 15A shows the relative configuration of each component of the microchip as affixed to the skull: “m” denotes the motion sensor, “g” denotes green LEDs for the source of electromagnetic measurement radiation, “b” a blue LED for the source of electromagnetic stimulus radiation and “h” denotes the heat stimulus. FIG. 15B shows the reference frame for the roll, yaw, and pitch measurements on the mouse and shows the differences in these measurement directions without heat stimulus (“normal exploration”) and with heat stimulus (“heat stimulation”) where movement is highly coordinated as the animal shakes its head. FIG. 15C shows the motion sensor responses as measured following multiple heat stimuli of increasing temperature and duration showing threshold of response and severity of response.

FIG. 16 (FIGS. 16A-16E) provides representative analysis of heart rate in a single mouse. FIG. 16A provides a trace of a skull-attached OIS microchip (top) and ECG (bottom) recording under anesthesia. The OIS waves match the ECG (dotted lines) showing the relationship between heartbeat and OIS. The OIS reading also shows a rolling average over a 0.5 s window emphazing a slower wave in the OIS which lines up with an artefact in the ECG caused by breathing of the animal (dotted lines marked with “*”). FIG. 16B provides a section of the OIS/ECG recording during a premature ventricular contraction, further confirming OIS has a component that follows heart activity. FIG. 16C is a tract of a skull-attached microchip OIS recording showing low-amplitude oscillations that are indicative of the arterial pulse. FIG. 16D provides the Fourier Transform of this signal, illustrating the frequency of the low-amplitude oscillations in FIG. 16C are consistent with heart rate. FIG. 16E provides comparative traces of OIS (top) with heart rate (bottom).

FIG. 17 (FIGS. 17A and 17B) provides the differences in CSD thresholds in the awake vs. sleep states. Multiple CSDs were triggered when the animals were sleeping or awake for different durations (21 awake, 26 asleep in 6 animals). The duration of blue light stimulation required to trigger CSD was taken as the CSD threshold. FIG. 17A shows the results when animals were awake (left), the average stimulation duration per animal required to trigger CSD was significant longer than when animals were asleep (red, n=6, p=0.037). FIG. 17B provides the stimulation durations of all CSD (n=47) triggered in all animals plotted as a function of how long the animal was in the sleep vs. wake state. The CSD threshold and sleep/awake duration are moderately correlated, i.e. the longer an animal is asleep the lower the CSD threshold, while the longer an animal is awake the higher the CSD threshold.

FIG. 18 (FIGS. 18A-D) provides measured characteristics and analysis of CSD in the wake state vs. the sleep state. This analysis compares 2 CSDs in each of 6 animals, one triggered in the fully awake state, and one triggered in the fully asleep state. “Fully,” in the context of these experiments, indicates that the animal was in that state before the CSD and throughout the CSD. FIG. 18A provides traces showing averages of all CSDs analyzed in the sleep and wake states, as well as a single trace of a CSD under anesthesia for comparison. The bands surrounding each trace indicate the standard deviation (n=6). The CBV response to CSD had clearly different shapes, amplitudes, and durations in each state. FIG. 18B provides representative traces of OIS (CBV) changes with CSD triggered in the awake vs. sleep states. CSD is associated with a multiphasic decrease in CBV, including an initial propagated wave of decreased CBV (Phase I) followed by a sustained global decrease in CBV (Phase IIa and IIb). For analysis purposes, 5 different phases of CBV were distinguished: baseline, Phase I, Phase IIa, Phase IIb and post (exemplified by the shaded regions in the figure). The border between phase I and IIa is defined as the max recovery after Iₘₐₓ in the sleep state (closed star in sleep state). The point of maximal recovery in the wake state (open star) is the border between IIa and IIb. The maximal decrease in CBV in phase I is indicated as Iₘₐₓ (closed arrowhead), whereas maximal CBV decrease associated with Phase II is indicated by IIₘₐₓ (open arrowhead). CSD triggered in the awake state, Phase IIa is characterized by a recovery toward baseline (open star) which is often hard to observe in the sleep state. FIG. 18C provides boxplots of the amplitudes of the distinct phases of awake and sleep CSD (both ANOVAs p<0.001). The maximal decrease in CBV in phase I (Iₘₐₓ) in sleep CSD is significantly greater than awake CSD. The maximal CBV decrease in Phase II (IIₘₐₓ) is equal in both awake and sleep CSD but shows a significant partial recovery in phase IIb with awake CSD but not sleep CSD. The other CSD phases were not significantly different in the wake vs. sleep states (*p<0.05, **p<0.01). FIG. 18D provides box plots illustrating the duration of CSD in the wake vs. sleep states and shows that the entire neurovascular response to CSD triggered during sleep lasts significantly longer than CSD is triggered in the awake state (p=0.012).

FIG. 19 (FIGS. 19A and B) provide analysis and correlation of CSD characteristics with time in the wake state vs. the sleep state. FIG. 19A shows four representative CSDs of one animal plotted sequentially, ordered by the duration the animal was in the wake or sleep state. The sequence shows the graded differences in the kinetics of the CBV response to CSD, including the maximal decrease in CBV of Phase I (Iₘₐₓ, filled arrowheads) and the maximal CBV decrease associated with Phase II (IIₘₐₓ, orange open arrowheads). FIG. 19B shows the measured OIS amplitudes of all CSDs (n=47) of all animals (n=6) as a function of how long the animal was in the wake or sleep state before CSD induction. Both Iₘₐₓ and Phase IIb amplitude are moderately but significantly correlated with the time an animal was in the wake or sleep state before CSD induction, corroborating the observations shown in FIG. 19A. Dotted lines are the best linear fit (trendlines) purely for visual reference. For statistical analysis, no linear relationship was assumed.

FIG. 20 (FIGS. 20A and B) show analysis of subject movement during CSD (n=6 animals). FIG. 20A is an exemplary trace of movement (lower trace, upper is OIS for reference) during sleep and awake CSD. Movement is measured as the angular momentum of the head relative to the average angular momentum while the animal is wake state throughout the whole experiment. With CSD evoked while the animal is in the wake state (left, bottom) animals showed reduced movement (freezing; open arrowhead, black overlay) during Phase IIa. CSD evoked while the animal is in the sleep state (right) caused transient awakening as indicated by a brief period of movement (filled arrowhead, black overlay), after which the animal returned to sleep until the recovery of the CBV to baseline, at which time the animal awakened and showed increased movement. FIG. 20B provides Tukey boxplots of movement during each phase of the CSD. Movement was compared between the average baseline level, the 30 second window with maximal movement change observed during Phases I and IIa (30 s max movement), the average movement in Phase IIb and the post-CSD phase. These plots illustrate a significant transient increase in movement with awakening when CSD is triggered during sleep compared with a significant transient decrease in movement consistent with freezing behavior when CSD is triggered in the awake state (**p<0.01).

FIG. 21 (FIGS. 21A and B) provide heart rate (HR) analysis during CSD (n=5 animals). FIG. 21A is a comparison of HR at the different phases of CSD. CSD triggered in the awake state was associated with a significant decrease in HR in Phase IIa, whereas CSD triggered in the sleep state was associated with a significant increase in HR during Phase I. FIG. 21 provides distribution density plots show that there is a strong positive correlation between HR and movement (angular momentum, ω) at baseline and phase IIb, whereas there is a weak to moderate inverse correlation between heart rate and movement during phase I/IIa) and post CSD. These results indicate that CSD disrupts the normal correlation between HR and movement, suggesting an autonomic response to CSD.

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%.

As used herein, the term “subject” refers to any organism which may be studied in measurement protocols designed to assess behavior. In some embodiments, the subject may be an organism which neural activity may be monitored via OIS and/or fluorescence (e.g., subjects expressing neural tissue with one or more fluorophores). Typical subjects include any animal (e.g., mammals such as mice, rats, rabbits, non-human primates, and humans). In some embodiments, subject is a rodent model of disease (e.g., a rodent model of sleep disorders, Alzheimer's disease, diabetes, or heart disease). In some embodiments, the subject is a rodent expressing opsins like channelrhodopsin or halorhodopsin.

The “recording devices” of the present disclosure includes those devices capable of storing the signals received from a microchip and/or microchip system. The recording device may include one or more data storage devices including microcontrollers, microprocessors, computers, servers, computer memory, semiconductor memory (e.g., memory cells build from MOS transistors), flash memory, ROM, PROM, EPROM, EEPROM, DRAM, or SRAM. The recording device may receive the raw signals from the sensors individually and independently. In some embodiments, the OIS measuring microchip comprises a recording device (or “microchip recording device”) such as EEPROM which each signal to and from one or more (e.g., each) component may pass therethrough prior to reaching a recording device not located on the microchip. In some embodiments, the system comprises a recording device external from the microchip. In some embodiments, the system comprises a recording device external from the microchip and a microchip recording device.

As used herein, “microchip” may refer to integrated circuits and/or microelectronics. A microchip may comprise a substrate, any indicated component, and an integrated circuit which transmits and/or receives electrical signals from that component. A “microchip system” refers to one or more microchips as indicated. Each microchip in a microchip system may have separate integrated circuits. In some embodiments microchips are connected together via flexprint.

Microchips of the present disclosure may be flexible and designed to conform to the skull. Flexible microchips are disclosed in WO 2002/055058A2 which is hereby incorporated by reference in its entirety. Each of the components of the present disclosure that are on the microchip or microchips (e.g., the optical sensor, the motion sensor, the heat stimulus, the source of electromagnetic measurement radiation, the source of electromagnetic stimulus radiation, the transmitters) are connected to the integrated circuit of the microchip or microchips via a flexible supporting layer attached to a surface of the device elements. The flexible supporting layer can, for example, comprise a polymer, such as a polyimide, polyester, parylene, or hydrogel. The flexible supporting layer typically is attached the microchip device elements on the side distal the release/exposure opening of the reservoirs (i.e. the release side). Alternatively, the microchip device elements could be effectively imbedded within the flexible supporting layer. In some embodiments, the flexible connections within the circuit comprise one or more hinges or flexible tethers connecting two or more of the device elements.

The microchip or microchips may be attachable to the skull (e.g., reversibly attachable to the skull such as with adhesives including super glue or acrylic based adhesives such as cyanoacrylate. Typically, the adhesive does not interfere with the detection of any parameter (e.g., neurovascular activity) to the skull of said subject. The microchip or microchips may be dimensioned such that it comprises a perimeter (e.g., a raised edge) to allow for the microchip or microchips to be adhered to the skull. In embodiments, the adhesive may comprise an adhesive selected from medical adhesive, tissue adhesive, or surgical adhesive. In some embodiments, the medical adhesive is selected from acrylic adhesives, silicone-based adhesives, hydrogel adhesives, and synthetic elastomeric adhesives. In further embodiments, the adhesive is a cyanoacrylate polymer. In certain aspects, the cyanoacrylate polymer is selected from n-butyl-2-cyanoacrylate and isobutyl cyanoacrylate. In various implementations, the adhesive coating comprises fibrin glue. In some embodiments, the adhesive coating comprises a bioactive film. In some embodiments, the adhesive comprises a pressure sensitive adhesive such as a pressure-sensitive acrylic adhesive. In some embodiments, the adhesive may comprise a rubber-based adhesive.

Affixing or attaching any of the microchips of the present disclosure to the skull of the subject may occur at one or more positions on the skull chosen for a specific area to be monitored and/or stimulated. The microchips may be attached directly to the skull (e.g., by parting the skin in a specific area and cleaning the skull to form an area prepared for attachment) by applying a layer of adhesive (e.g., cryanoacrylate) over the prepared skull and the sensors/stimulus on a microchip are applied directly thereto. Following drying of the adhesive, a layer of filler such as dental cement or epoxy may be applied in the cavities formed between the microchip and skull. Particularly in embodiments involving optical sensors, this filler layer may cover the sources of light and/or optical sensors in order to prevent or reduce the electromagnetic radiation other than the measurement radiation from being measured by the optical sensor. In some embodiments, following hardening of this first filler layer, the entire microchip may then be covered with a second filler such that only the connector on the microchips is not covered by the filler.

Microchips may have a chip architecture configured to allow transmission of the sensor data to a recording device configured to record this data. Typically, transmission occurs through a connector on one or more microchips in the microchip system. The recording device may be, for example, a computer, microcontroller, solid state memory storage. In some embodiments, the chip architecture is also configured to transmit, from an external source such as microcontroller, appropriate signals to the components of the chip (e.g., the source of electromagnetic measurement radiation, the source of electromagnetic stimulus radiation, the heat stimulus, the optical sensor, the pressure sensor, the motion sensor) to allow their independent operation. In some implementations, the micro-controller is programmed to automatically deliver a stimulus in response to specific characteristics of data from the sensors or camera (e.g., by sending a current through a resistive heater on a heat stimulus microchip when the subject is located in a specific area of the enclosure as measured by the camera and/or the motion sensors have transmitted data corresponding to a sleep/awake cycle). In various implementations, the microchips are controlled via a server configured to interface with a microcontroller and the microcontroller is configured to interface with the microchips. In some embodiments, the microchips comprise a connector for a wired connection between said microchip and said recording device. In various implementations, the microchips further comprise a battery connected to the source of electromagnetic measurement radiation and/or the source of electromagnetic stimulus radiation and/or the optical sensor and/or the motion sensor and/or the thermal sensor and/or the pressure sensor and/or the heat source. In some embodiments, the microchip comprises radio-frequency identification components.

A) Spectral Microchips (e.g., Spectral Measurement Microchips, Spectral Stimulus Microchips)

Microchips of the present disclosure are typically used for the detection of neurovascular activity in a subject (e.g., mouse, rat, primate, human, dog). The microchips of the present disclosure may be a piece made of glass or plastic, with the size of a few millimeters (e.g., 1-10 mm, 1 mm-100 mm) (length)×a few millimeters (e.g., 1 mm-10 mm, 1 mm-100 mm) (width) and a few tenths of millimeters (e.g., 0.1 mm-1 mm) in height. The microchips of the present disclosure generally include those with integrated circuits and microelectromechanical systems to perform the indicated stimulus and/or measurement functions. Any microchip in the present disclosure my further include passive radio-frequency identification (RFID) technology, also known as a PIT (passive integrated transponder) tag. The microchip may be generated from metal-oxide-silicon (MOS). In some embodiments, the microchip is flexible microchip.

The microchip and/or microelectronics system may comprise:
a) a source of electromagnetic measurement radiation (or a first source of electromagnetic radiation) arranged on said microchip system such that when said microchip system is attached to said skull, at least a portion of said electromagnetic radiation from said source reflects off of and/or elicits fluorescence from neural tissue underlying (and/or fluorescent molecules therein) said skull; and
b) an optical sensor arranged on said microchip such that when said microchip is attached to said skull, said optical sensor is configured to sense a portion of said reflected electromagnetic radiation off of or fluorescence from neural tissue (and/or fluorescent molecules therein) underlying the skull;
wherein changes in the reflected electromagnetic radiation and/or fluorescence correlates with said neurovascular activity; and
wherein said microchip is configured to receive signal from said optical sensor and said microchip is configured to transmit an electrical signal corresponding to at least one property of said electromagnetic radiation. In some embodiments, the electromagnetic radiation comprises green light and/or infrared light. For example, the source of electromagnetic measurement radiation may comprise a light emitting diode (or “measurement light emitting diode”). The light emitting diode may emits a spectrum of electromagnetic radiation having a λₘₐₓ from 275-950 nm.

The light source in the spectral microchips of the present disclosure (e.g., Spectral Measurement Microchips, Spectral Stimulus Microchips) may include any suitable light source. In some cases, the light source is an LED, an LED array or a laser. The light source may emit light having a wavelength in the infrared range, near-infrared range, visible range, and/or ultra-violet range. In some cases, the light source may emit a light at a wavelength in the range of 350 nm to 2,000 nm, e.g., 410 nm to 2,000 nm, 440 nm to 1,000 nm, 440 nm to 800 nm, including 440 nm to 620 nm. The light source may be configured to produce a continuous wave, a quasi-continuous wave, or a pulsed wave light beam.

In some embodiments, the source of electromagnetic radiation comprises (or consists of) one or more light emitting diodes independently having an output power of less than (or from 0.01 μW/cm² or 0.1 μW/cm² to) 500 μW/cm² (e.g., from 1 μW/cm² to 200 μW/cm², from 1 μW/cm²-50 μW/cm², from 1 μW/cm²-20 μW/cm², from 1 μW/cm²-10 μW/cm², from 1 μW/cm²-30 μW/cm², from 20 μW/cm²-30 μW/cm²,). In some embodiments, the source of electromagnetic measurement radiation comprises or consists of from 1-10 light emitting diodes (e.g., 1, 2, 3, 4, 5, 6, 7, 8, 9 10, from 1-5, from 2-4). In some embodiments, the source of electromagnetic measurement radiation has a light output of from less than (or from 0.01 μW/cm² or 0.1 μW/cm² to) 500 μW/cm² (e.g., from 1 μW/cm² to 200 μW/cm², from 1 μW/cm²-50 μW/cm², from 1 μW/cm²-20 μW/cm², from 1 μW/cm²-10 μW/cm², from 1 μW/cm²-30 μW/cm², from 20 μW/cm²-30 μW/cm²,). In some embodiments, the source of electromagnetic measurement radiation has one or more (e.g., one, two, three) λₘₐₓ (e.g., each in the range from 275-950 nm, ultraviolet to visible, visible to infrared), wherein the light emitting sources that produce each individual λₘₐₓ may be independent controlled. For example, the source of electromagnetic measurement radiation may have one or more spectral maximums λₘₐₓ (e.g., each in the range from 500-600 nm).

The spectrum of said source of electromagnetic measurement radiation may be tuned for OIS and/or fluorescence measurements (and, particularly, OIS measurements measuring reflectance off of the intracranial neural tissue of the subject). In some embodiments, the reflected light is transmitted directly to the optical sensor (e.g., there are one or more windows in the skull for between radiation transmission from the source and measurement by the optical sensor). In some embodiments, the measurement radiation passes through the skull between the source and the neural tissue and/or the reflected radiation passes through the skull prior to detection by the optical sensor.

The spectral microchips of the present disclosure may also be configured for elicitation of fluorescence of a fluorescent molecule expressed within or introduced into the neural tissue. In some embodiments, the fluorescent light is transmitted directly to the optical sensor through one or more windows in the skull for transmission. In some embodiments, the measurement radiation passes through the skull between the source and the neural tissue prior to absorption. In some embodiments, the fluorescence radiation passes through the skull prior to detection by the optical sensor. Fluorescent molecules may include genetically encoded ion indicators (such as the calcium indicator GCaMP), genetically encoded fluorescent proteins such as green fluorescent protein (GFP), or fluorescent nanoparticles introduced into the vasculature. In some embodiments, neurons of the neural tissue may comprise one or more neural activity-dependent fluorescent moieties such as those described in US Pub No 2020/0187780, which is hereby incorporated in reference in its entirety and particularly in relation to fluorescent molecules and moieties. The one or more neural activity-dependent fluorescent moieties may include a genetically-encoded indicator dye.

The one or more neural activity-dependent fluorescent moieties may include a calcium- and/or a voltage-sensitive indicator dye (and the source of measurement radiation may induce fluorescence from such dyes). In any embodiment, the one or more neural activity-dependent fluorescent moieties may include a calcium- and/or a voltage-sensitive indicator dye. Fluorescent moieties that may be used include those with fluorescence properties sensitive to cellular electrical activity such as ratiometric/non-ratiometric dyes and fluorescent proteins. In some embodiments, the fluorescent moieties may be a fluorescence resonance energy transfer (FRET)-based reporter. In some embodiments, the fluorescent moiety may be sensitive to changes in intracellular concentration of ions such as calcium, sodium and protons or to changes in membrane potential. In such cases, fluorescent dyes may be calcium indicator dyes (Indo-1, Fura-2, and Fluo-3, Calcium Green®, Fluo-4, etc.); sodium indicator dyes (sodium-binding benzofuran isophthalate (SBFI), Sodium Green™, CoroNa™ Green, CoroNa™ Red, etc.); and proton indicator dyes (2′,7′-bis-(carboxyethyl)-5-(and-6)-carboxyfluorescein (BCECF), etc.).

Additionally, the subject may express certain genetically encoded ion indicators and the spectral measurement microchip may comprise a light source to induce fluorescence of those indicators. For example, the subject may express one or more cellular electrical activity-dependent fluorescent proteins such as genetically encoded calcium indicators (Cameleon, Pericam, TN-XXL, Twitch, GECO, GCaMP1, GCaMP2, GCaMP3, GCaMP6 and derivatives thereof, as well as those cited in U.S. Pat. No. 8,629,256, and Tian et al. 2012 Prog Brain Res, 196:79, which are incorporated herein by reference); and genetically encoded voltage indicators (QuasAr1, QuasAr2, VSFP, and derivatives thereof, as well as those cited in US App. Pub. No. 2013/0224756, Hochbaum et al., Nat Methods 2014 11:825, Baker et al. Brain Cell Biol 2008 36:53; and Mutoh et al., Exp Physiol 201196:13, each of which are incorporated herein by reference). Other suitable GCaMP-based genetically encoded calcium indicators include GCaMP2.1, GCaMP2.2a, GCaMP2.2b, GCaMP2.3, GCaMP2.4, GCaMP3, GCaMP5g, GCaMP6m, GCaMP6s, GCaMP6f, etc. Suitable GECO-based genetically encoded calcium indicators include G-GECO1, G-GECO1.1 and G-GECO1.2, the red fluorescing indicator R-GECO1, the blue fluorescing indicator B-GECO1, the emission ratiometic indicator GEM-GECO1, and the excitation ratiometric GEX-GECO1.

The optical sensor may have a sample rate of less than (or from 1 Hz to) 20 kHz (e.g., from 100 Hz to 20 kHz, from 100 Hz to 10 kHz, from 100 Hz to 5 kHz, from 100 Hz to 2 kHz, from 200 Hz to 20 kHz, from 200 Hz to 10 kHz, from 200 Hz to 5 kHz, from 200 Hz to 2 kHz, from 500 Hz to 20 kHz, from 500 Hz to 10 kHz, from 500 Hz to 5 kHz, from 500 Hz to 2 kHz, from 100 Hz to 5 kHz, from 128 Hz to 5 kHz). The optical sensor may, for example, measure intensity of the reflected light to detect the changes in the neurovascular activity (e.g., cortical blood volume) of the subject.

The optical sensor may be configured to transmit the measurements to a microcontroller or processor (e.g., via Inter-Integrated Circuit (I²C) communication or via Serial Peripheral Interface (SPI) communication, through the integrated circuits of the microchip) for recording of the optical sensor measurements. In some embodiments, the microchip may be configured to measure and transmit information relating to one or more of the brain blood flow, respiration, heart rate, and movement. Such measurements may be performed for more than (or up to 50 sleep-wake cycles) one sleep-wake cycle of the subject (e.g. more than two sleep-wake cycles, more than five sleep-wake cycles more than ten sleep-wake cycles, more than fifteen sleep-wake cycles, more than twenty sleep-wake cycles, more than twenty-five sleep wake cycles). In some embodiments the measurements may be performed for a period of one day to six months or from 1 day to 30 days or from 1 week to 6 months or from 1 week to 1 month or from 1 day to 2 weeks. Measurements, and particularly spectral measurements such as OIS and/or fluorescence measurements, may occur over the entirety or portions of these time periods with a frequency of, for example, less than (or from 1 Hz to) 20 kHz (e.g., from 100 Hz to 20 kHz, from 100 Hz to 10 kHz, from 100 Hz to 5 kHz, from 100 Hz to 2 kHz, from 200 Hz to 20 kHz, from 200 Hz to 10 kHz, from 200 Hz to 5 kHz, from 200 Hz to 2 kHz, from 500 Hz to 20 kHz, from 500 Hz to 10 kHz, from 500 Hz to 5 kHz, from 500 Hz to 2 kHz, from 100 Hz to 5 kHz, from 128 Hz to 5 kHz). The optical sensor typically measures the intensity of coming light, and particularly, that light reflected off of the neural tissue underlying the skull of the subject, or fluorescence elicited from a fluorescent molecule within the neural tissue.

In some embodiments, the microchip comprises a filter configured such that radiation other than the radiation to be measured (e.g., radiation not having a wavelength of OIS measurement, radiation not having a wavelength of the fluorescence excitation/emission) is filtered out (e.g., the radiation intensity of non fluorescent radiation entering the optical sensor is decreased or removed). Suitable optical sensors include photoresistors or photovoltaics, commonly known as solar cells, convert an amount of incident light into an output voltage, and photodiodes such as phototransistors.

Also provided are spectral probe microchips which may be configured to induce a response in the subject (such as a subject expressing opsins like channelrhodopsin or halorhodopsin). These spectral probe microchips typically direct electromagnetic radiation toward the neural tissue in order to elicit a response from the subject.

The microchip may comprise a source of electromagnetic stimulus radiation (or second source of electromagnetic radiation) which is used to illicit a response in the subject. For example, the microchip may further comprise a source of electromagnetic stimulus radiation; wherein the electromagnetic stimulus radiation is electromagnetic radiation emitted from said source towards the neural tissue underlying said skull (e.g., through the skull); and wherein said stimulus electromagnetic radiation induces a response in said subject. In some embodiments, the electromagnetic stimulus radiation may be used to induce cortical spreading depression (CSD) in a subject by transmitting light to the neural tissue of subjects which would have such a response. For example, the subject may be a subject expressing channelrhodopsin or halorhodopsin. In various implementations, the source of electromagnetic stimulus radiation comprises a light emitting diode (or “stimulus light emitting diode”).

The stimulus light emitting diode may emit a spectrum of electromagnetic radiation having one or more spectral maximums λₘₐₓ from 300-500 nm. In some embodiments, the stimulus light emitting diode has an output power of less than (or from 0.01 μW/cm² or 0.1 μW/cm² to) 500 μW/cm² (e.g., from 1 μW/cm² to 200 μW/cm², 1 μW/cm² to 50 μW/cm², 10 μW/cm² to 50 μW/cm²). In various implementations, the source of electromagnetic stimulus radiation comprises (or consists of) from 1-10 light emitting diodes (e.g., 1, 2, 3, 4, 5, 6, 7, 8, 9 10, from 1-5, from 2-4).

The sources of electromagnetic radiation (e.g., the source of electromagnetic measurement radiation, the source of electromagnetic stimulus radiation) may comprise one or more light emitting diodes (LED) (e.g., one or more AlN LEDs, BN LEDs, diamond LEDs, GaN LEDs, AlGaN LEDs, InGaN LEDs, Al,GaINP LEDs, AlGaAs LEDS, combinations thereof), lasers, or excimer lamps. The sources may have one or more local spectral maximum such as one or more in the range from 600 nm to 1100 nm or from 400 nm to 600 nm. Each local spectrum having a spectral maximum wavelength may independently have a full-width half maximum of less than 50 nm or less than 40 nm or less than 20 nm or less than 10 nm or less than 5 nm or less than 1 nm. The LED may be, for example a green LED (e.g., when used in the source for electromagnetic measurement radiation) or a blue LED (e.g., when used in the source for electromagnetic stimulus radiation).

B) Behavioral Measurement Microchips

The present disclosure also includes microchips comprising one or more sensors for the measurement of other behaviors in the subject. Such microchips include one or more of:
a) a motion sensor; or
b) a thermal sensor for the measurement of skull surface temperature of the subject;
c) a pressure sensor for the measurement of intracranial pressure of the subject.

For example, the microchips of the present disclosure may comprise one or more (e.g., one, two, three, four, five, six, seven, eight) motion sensors. The motion sensors may comprise one or more accelerometers. The motion sensors may be configured to measure movement of the skull in a particular direction and/or acceleration of the skull. The motion sensors may transmit data in order to identify motion in all three directions of a reference frame like acceleration (e.g., aX, aY, and aZ) and/or rotation (e.g., ωX, ωY, ωZ) in all three directions. The reference frame for acceleration and position may be the same or different. In some embodiments, the motion sensor comprises a magnetometer.

Microchips of the present disclosure may comprise additional sensors to allow for additional and potentially concurrent measurements with the spectral measurements and/or motion/acceleration measurements. For example, the microchip may comprise a thermal sensor (e.g., thermometer, thermocouple, thermistor) for measurement of skull surface temperature, a pressure sensor (e.g., piezoresistive pressure sensors) for measurement of intracranial pressure. Microchip systems involving thermal measurement microchips may be particularly well suited for track temperature changes associated with the estrous cycle of the subject under study. Temperature fluctuations as measured by the thermal sensor may be correlated with, for example, hormone measurements taken from vaginal swabs of the subject under study.

C) Stimulus Microchip

Additionally, the present disclosure includes microchips which may be affixed to the skull in order to provide additional stimulus to the subject enders study. The microchip may comprise one or more components to stimulate the subject (in a manner different than with the electromagnetic stimulus radiation directed towards the skull) and consequently measure reactions to the stimulus with the other sensors described herein. For example, in some embodiments, the microchip may comprise a heat source (e.g. a resistive heater). The heat source may be used to deliver a calibrated painful stimulus or measure heat-pain thresholds in concert with the other measurements performed. For example, the heat stimulus may have an operating temperature of from room temperature to 200° C. or 150° C. or 120° C. In some embodiments, the heat stimulus is a resistive heater.

Microchip Systems

Any of the microchips of the present disclosure (e.g., spectral microchips such as the spectral measurement microchips, spectral stimulus microchips, behavioral measurement microchips such as the motion sensor microchips, the thermal sensor microchips, pressure sensor microchips, stimulus microchips) may be integrated together on the same integrated circuit (e.g., a microchip may comprise a spectral measurement microchip and a spectral stimulus microchip). In some embodiments, each set of components are present on a substrate and have their own integrated circuit. In various embodiments, several microchips are connected via “flexprint” in order to form a microchip system comprising two or more microchips of the present disclosure (e.g., spectral microchips such as the spectral measurement microchips, spectral stimulus microchips, behavioral measurement microchip such as the motion sensor microchips, the thermal sensor microchips, pressure sensor microchips, stimulus microchips).

The microchips, microchip systems, systems, and methods of the present disclosure are less invasive than other methodologies and allow for free movement of subjects under study. In various embodiments, the microchip has a weight of less than (or from 0.1 g to) 10 g (e.g., less than 5 g, less than 2 g, from 0.5-2 g, from 1-3 g).

The microchip for the detection of neurovascular activity in a subject may comprise:
a) a means for affixing said microchip to the skull of said subject (e.g., via an adhesive such as an acrylate-based adhesive);
b) a first means for emitting electromagnetic radiation to be reflected off of or elicit fluorescence from neural tissue (and/or fluorescent molecules therein) underlying the skull of said subject (e.g., one or more light emitting diodes, a laser);
c) a sensor means for sensing electromagnetic radiation reflected off of and/or fluorescence from the neural tissue (or fluorescent molecules therein) underlying the skull of said subject; wherein changes in the reflected radiation and/or fluorescence are sensed by the sensor means correlate with said neurovascular activity (e.g., photoconductive devices, photodiodes, photovoltaics, phototransistors); and
d) a transmission means for transmitting an electrical signal corresponding to the radiation measured by the sensor means (e.g., wireless transmitters integrated with the chip architecture for receipt and/or transmission of electrical signals, wired connectors integrated with the chip architecture for receipt and/or transmission of electrical signals).

A microchip for the detection of behavior in a subject may comprise:
a) a means for affixing said microchip to the skull of said subject (e.g., via an adhesive such as an acrylate-based adhesive);
b) a sensor means for detection of the motion, temperature, or pressure of the subject and;
c) a transmission means for transmitting an electrical signal corresponding to the radiation measured by the sensor means (e.g., wireless transmitters integrated with the chip architecture for receipt and/or transmission of electrical signals, wired connectors integrated with the chip architecture for receipt and/or transmission of electrical signals).

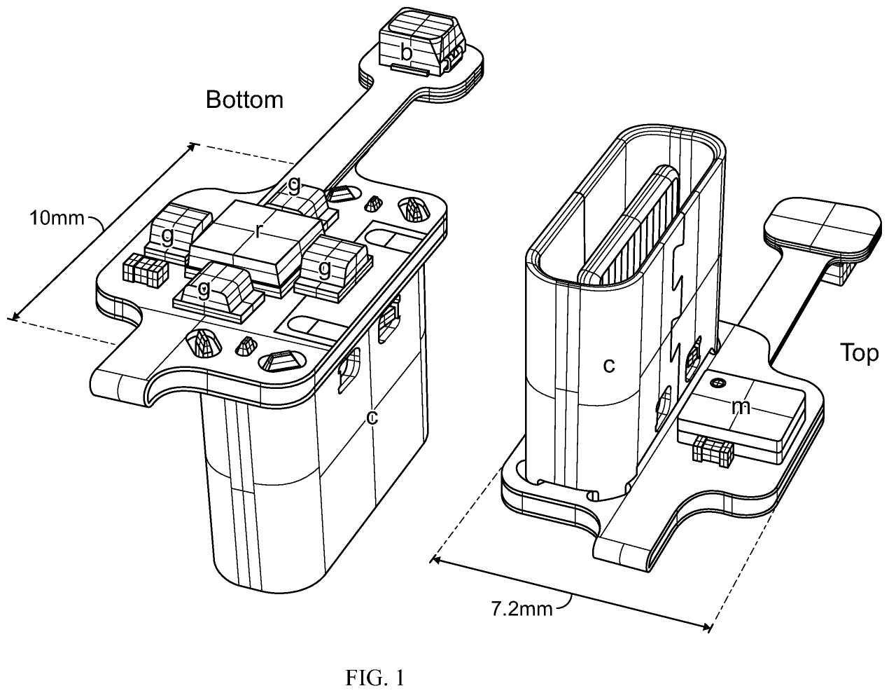

FIG. 1 provides a prospective view of an exemplary microchip system for performing OIS measurements and providing stimuli to the subject. The microchip portion affixed toward the skull (or “bottom” of the microchip) has maximum lengths from 5-15 mm. As can be seen, the microchip system comprises IOS chip architecture integrated with the optical sensor (r), a source of electromagnetic measurement radiation which is four green LEDs surrounding the optical sensor (g), a source of electromagnetic stimulus radiation (b) which is a blue LED in this example. The blue LED may be placed at a variety of positions in relation to the optical sensor due to the bridge flexes on the chip architecture. On the other side of the microchip (or the “top” which is oriented away from the skull when affixed), the microchip further comprises a motion sensor (m) and a connector (e.g., a USB connector such as USB-C) for transmitting the measurements from the optical sensor and the motion sensor and/or transmitting power to the sources of electromagnetic radiation.

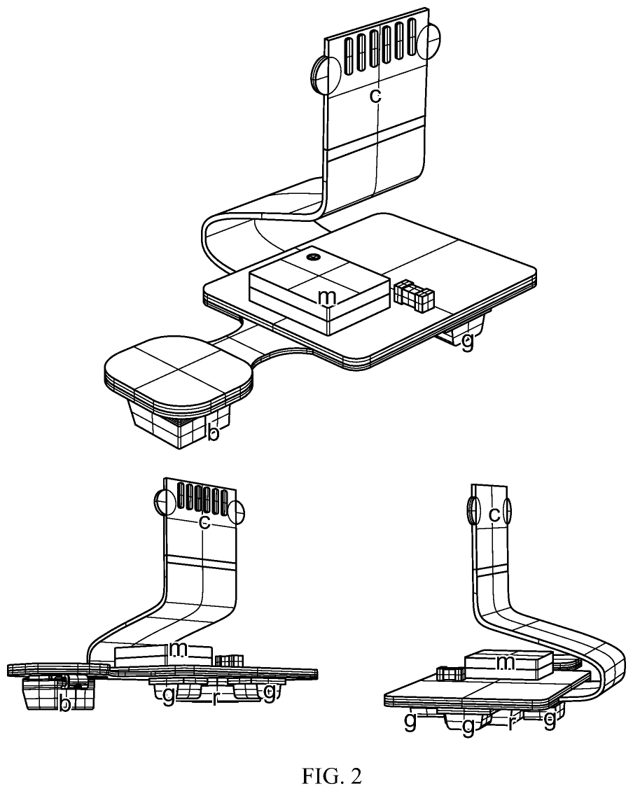

Referring now to FIG. 2, a similar microchip system is illustrated comprising the optical sensor (r), a source of electromagnetic measurement radiation which is four green LEDs surrounding the optical sensor (g), a source of electromagnetic stimulus radiation (b) which is a blue LED in this example and a motion sensor (m). The microchips in the depicted system are integrated through a flexprint to a microcircuit board shape (c, either flexible or not) that can be to be connected with a connector such as an FFC/FPC connector which may be used to connect the microchip system to a recording device capable of receiving signals from the sensors.

The microchips may be coupled to individual substrates and coupled together by a flexible printed circuit board (or “flexprint”). The substrate and the flex can include any suitable wiring and routing to electrically couple the die to other parts of the system such as, for example, a flash memory. In some embodiments, the flex can be coupled to a different surface of the substrate than the individual components in any microchip or die. In some embodiments, the flex can be coupled to the same surface of the substrate as the die. In some embodiments, the flex can include a ledge to which one or more components can be coupled.

In some embodiments, rather than being included together in a single die, the components of a system can be included as discrete entities. The discrete entities can be coupled to a substrate rather than being coupled to a printed circuit board (“PCB”). As a substrate can have more stringent design rules than a PCB, coupling the discrete entities to the substrate can allow for a system that is smaller and more compact in size. For example, the wiring for the system can be created using less layers and can be formed more densely in a substrate than in a PCB. Such couplings between microchips and flex in a microchip are disclosed in U.S. Pat. No. 8,334,704, which is hereby incorporated by reference in its entirety.

The individual components on the microchip (e.g., the optical sensor, the source of electromagnetic measurement radiation, the source of electromagnetic stimulus radiation, the motion sensor, the pressure sensor, the temperature sensor, the heat stimulus microchip) may each individually communicate on the microchip system with a memory device integrated on the microchip system as well to result in ultimate communication of the multiple data streams from the sensors and input signals to the components to a recording device separate from a microchip. In some embodiments, microchip comprises a microcontroller (e.g., electrically erasable programmable read-only memory) where one or more (including all) of the components interface with the microcontroller on the microchip. The microcontroller microchip, may interface with an external recording device for transmission, recording, and analysis of data. Table 1 describes several of the components which may be found on the microchip. The components may be integrated with flexible bridges between the components and the integrated circuits created thereby to afford flexibility of the microchip.

TABLE 1

| | | | | | Exemplary |
|---|---|---|---|---|---|
| | | | | | Maximal |
| CHIP SYSTEM | Communication | Sample rate | | Size | Resolution |
| Optical Sensor | I2C, 400 kHz | 0 to >=1024 | Hz | 2.8 × 2.8 × 1 mm | 7200 count at |
| | | | | (<8 mm³) | 10 uW/cm2 |
| | | | | | (571 nm) |
| Source of | | | | 2 − 4 × λₘₐₓ = 571 nm, | |
| Electromagnetic | | | | each ≤1 mm³ | |
| Measurement | | | | | |
| Radiation | | | | | |
| Source of | | | | λₘₐₓ = 460 nm | |
| Electromagnetic | | | | (<7 mm³) | |
| Stimulus | | | | | |
| Radiation | | | | | |
| Motion Sensor | I2C, 400 kHz | 12.5 to >=1666 Hz | | 2.5 × 3 × 0.86 mm | 4.375-70 mdps |
| | | (Angular); | | (<7 mm³) | (angular); |
| | | 12.5 to >=6664 Hz | | | 0.061 mg (linear), |
| | | (Linear); | | | 0.14 mgauss |
| | | 0.625 to >=80 Hz | | | (magnetometer) |
| | | (Magnetometer) | | | |
| Pressure Sensor | I2C, 400 kHz | 0 to >=70 | Hz | 3.3 × 3.3 × 2.9 mm | 6 mPa, accuracy |
| | | | | (<32 mm³) | 0.1 hPa |
| Temperature | I2C, 400 kHz | 0 to >=8 | Hz | <1 mm³ | At least 0.0625-1° |
| Sensor | | | | | C./LSB |
| EEPROM | I2C, 400 kHz | | | <1 mm³ | |
| Analogue | Serial | >=5 kHz | | | 5 μV |
| Physiology (e.g., | | (per channel) | | | |
| EEG/evoked | | | | | |
| potentials) | | | | | |

| | | | | |
|---|---|---|---|---|
| Heat Stim | Serial | heat feedback | <1 mm³ | temperature can |
| | | loop >=5 kHz | | be read out and |

corrected within
a resolution
of <=0.5° C.

Each of these components in Table 2 may be integrated onto a microchip or microchip system, wherein the microchip or microchip system is configured to be affixed to the skull of a subject. Decreased invasiveness may require decreased weight systems. In some embodiments, the microchip has a weight of less than (or from 0.1 g) to 10 g or less than 5 g or less than 3 g or less than 2 g (e.g., from 0.5 to 5 g, from 1 to 3 g, less than 1 g, from 0.5 g to 1 g). An exemplary microchip system includes one or more (or all) of:
an IOS sensing microchip system (e.g. comprising of sensor microchip and 2-10 LEDs);
a Motion sensing microchip;
an intercranial pressure sensing microchip;
EEPROM which includes storing information on the subject (e.g., age, weight, source, genotype, subject #, experimental settings)
a thermal measurement microchip system (e.g., from 1-8 thermal measurement microchips)
a heat stimulus microchip comprising, for example, 1-3 thermistors; and
open channels for simultaneous recording/triggering of other measurement components such as EEG/evoked potentials.

The microchips and microchip systems typically interface directly or indirectly with other components such as the recording devices (e.g., one or more computers, one or more microcontrollers, one or more servers, solid state storage, one or more displays) in a system for the detection of neural activity and/or behavior including neurovascular activity, motor activity, temperature, and intracranial pressure in one or more subjects. The systems for the detection of neurovascular activity in a subject may comprise:
a) a microchip configured to measure optical signal (e.g., OIS, fluorescent signal) while being affixed to the skill of a subject, and
b) a recording system in communication with the microchip, wherein each microchip transmits measurements from each sensor independently (e.g., measurements from the optical sensor, measurements, from the motion sensor, measurements from the thermal sensor, measurements from the pressure sensor, combinations thereof) to the recording system.

In some embodiments, the recording system is physically linked (e.g., through a wire) to the microchip. In various implementations, the recording system comprises a processor or a microcontroller which interfaces with said microchip; and the processor or microcontroller can receive measurement data from each sensor on the microchip and/or control components of the microchip (e.g., the source of electromagnetic measurement radiation and/or the source of electromagnetic stimulus radiation and/or the optical sensor and/or the motion sensor and/or the thermal sensor and/or the pressure sensor and/or the heat source). In some embodiments, the recording system comprises a microcontroller in communication with a server.

The measurement protocols and these systems of the present disclosure may afford the measurement of a freely-moving subject, particularly in an enclosure therefor. In some embodiments, the system comprises an enclosure for said subject (e.g., an enclosure where the subject can be monitored over the indicated time length such as more than (or up to 50 sleep-wake cycles) one sleep-wake cycle of the subject (e.g. more than two sleep-wake cycles, more than five sleep-wake cycles more than ten sleep-wake cycles, more than fifteen sleep-wake cycles, more than twenty sleep-wake cycles, more than twenty-five sleep wake cycles).

The system may further comprise a camera for monitoring the movement of said subject in said enclosure. In some embodiments, the enclosure comprises a flooring material offering a contrast in color with the subject such that the camera may continuously monitor subject position in the enclosure (and, correlate that position, for example, with the other data being measured such as the data from the optical sensor, the data from the motion sensor, the data from the pressure sensor, the data from the thermal sensor, or combinations thereof). For example, the system may comprise a microcontroller and optionally a camera, the microchip comprises a motion sensor, and the microcontroller is configured to identify awake and sleep cycles of said subject based on measurements transmitted thereto from said motion sensor and/or said camera. This data may be fed back into the system such that an optical and/or heat stimulus and/or other stimuli (e.g., changing of environmental parameters like daylight and/or barometric pressure) can be delivered automatically in response to data from one or more sensors and/or the camera, such as specific sleep/wake state or a specific location within the enclosure.

The system may comprise a master controller module having a one or more of: a microcontroller, a telemetry circuit, a server, a power module, a memory (preferably, a remotely programmable memory), a real-time clock, a bus (preferably a bi-directional bus), a plurality of signal modules which are connected to the bus, and circuitry for connecting to the individual channels of the microchip (e.g., via the interfacing connector with the microchip).

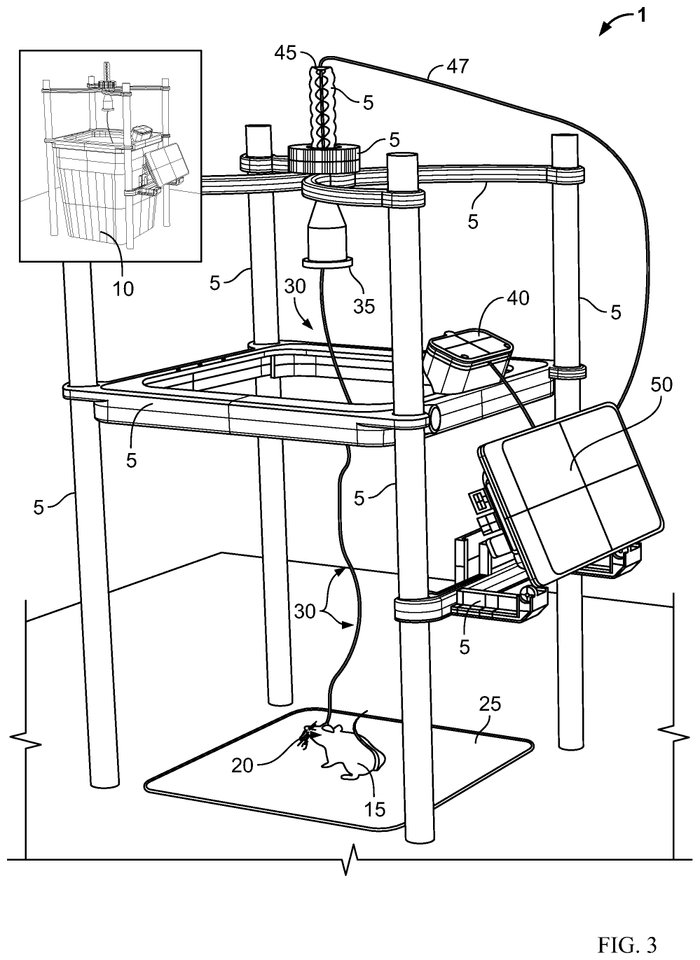

Referring now to FIG. 3, a system 1 is illustrated comprising microchip system 20 affixed to the skull of subject 15. System 1 comprises a support structure 5 and enclosure (see inset) 10 attached to allow for monitoring subject 15. Subject 15 is allowed to freely move around the base 25 of enclosure 10 during study. Microchip system 20 transmits measurements to a recording system through wires 30. Microchip system 20 may also receive signals from a processor such as a microcontroller via wires 30. Wires 30 enter commutator 35 to prevent rotation of wires prior to exiting support 5 at apex 45 for wires 47 to control/recording system/monitor 50. In some embodiments commutator 35 is a split-ring commutator. System 1 further comprises a camera 40 which may track the movement of subject 15 around enclosure 10. The base 25 (or bedding) may be chosen to provide a contrast with the subject facilitating such motion tracking via camera 40. In some embodiments, the system identifies when subject 15 is in a particular location on base 25 (e.g., via motion tracking afforded by camera 40) and the system is configured to deliver a stimulus (e.g., a heat stimulus) to the subject when the subject is in that location (e.g., via a thermal probe microchip). Measurements from microchip system 15 may be transmitted to a recording device (e.g., a microcontroller), which then transmits a visual signal of those measurements to one or more monitors 50.

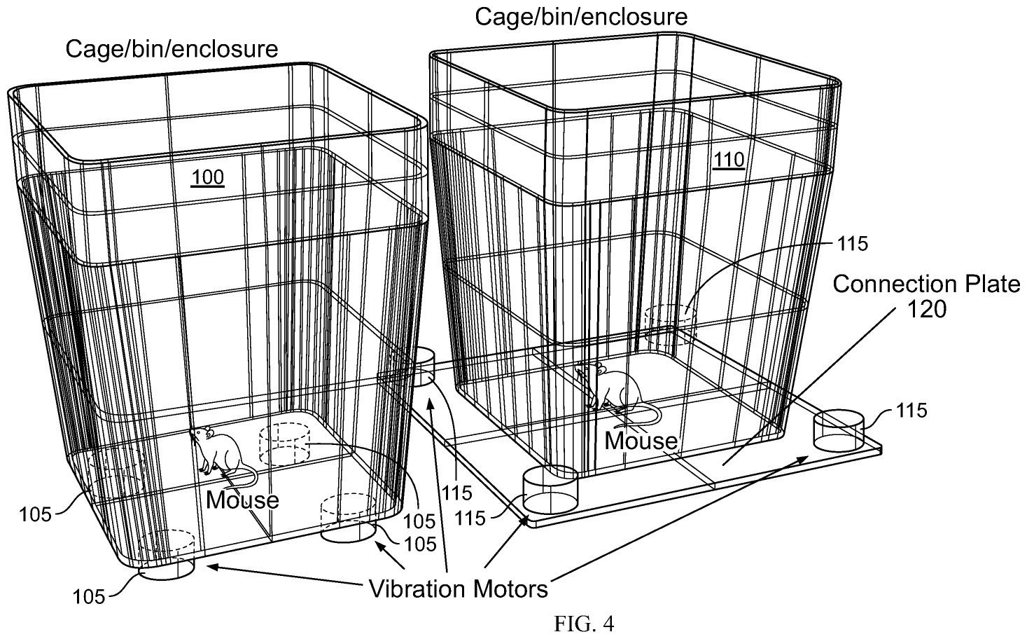

In some embodiments, the system may comprise a cage vibration system as well. Referring now to FIG. 4, a modification to the subject enclosure is illustrated. The cage vibration system may comprise one or more (e.g., one, two, three, four, five, six, seven, eight, nine ten) motors attached directly or indirectly to the enclosure (e.g., cage, bin). These motors may serve to stimulate a subject in the enclosure. For example, the motors may vibrate the enclosure with a force and for a time sufficient to induce an awake state of the subject. In FIG. 4, enclosure 100 has four vibration motors (each individually labelled 105) attached directly to the bottom of the enclosure and enclosure 110. In enclosure 110, the vibration motors (individually labelled 115) are attached indirectly to enclosure 110 via connection plate 120. The motors vibrate the connection plate (e.g., a solid unitary piece of material which the enclosure sits on such as plastic or metal) which causes vibration of the enclosure.

Applications

These microchips, systems, and methods of repetitive CSDs over weeks, may be used for characterization of the effects of migraine therapies, evaluation of the effects of the estrous cycle on CSD and sleep, and investigation of the effects of genetic mutations associated with migraine and sleep disorders. This disclosure may be used to investigate changes in brain activity, blood volume, heart rate, and behavior in a variety of physiological, pharmacological, and pathological contexts. These include investigation of other brain disorders like stroke, epilepsy, and neurodegenerative diseases, as well as a variety of systemic diseases in which long term, continuous recording of physiological and behavioral parameters may provide important new insights.

Methods for the detection of neurovascular activity in a subject having a microchip configured to measure optical signal (e.g., OIS, fluorescence) while being affixed to the skill of a subject are also provided, comprising:
a) emitting electromagnetic measurement radiation from a source of electromagnetic measurement radiation on the microchip to reflect off of the neural tissue and/or be absorbed by a fluorescent molecule therein underlying the skull of the subject; and
b) measuring the reflected electromagnetic measurement radiation and/or fluorescence with an optical sensor on the microchip;
wherein changes in the reflected electromagnetic radiation correlate with said neurovascular activity.

Recording of neurovascular activity and behavior may also assess changes associated with a neurological or systemic disease in a rodent model of such disease (e.g., a rodent model of sleep disorders, Alzheimer's disease, diabetes, or heart disease). Recording may also assess a subject response to certain pharmacological agents or therapeutic interventions such as a candidate drug substance, administration regimen, or device treatment. In some embodiments, the method further comprises administering to said subject a candidate drug substance or device treatment (e.g., a candidate drug substance administered once or with an administration regimen, device treatment which may have effects on brain neurovascular activity or sleep, candidate substances or device treatments for the treatment of cortical spreading depression or a disease, disorder, or condition associated therewith such as migraine aura, brain ischemia, or seizures). Using the systems of the present disclosure, the effects of the candidate treatment can be accurately determined by comparing brain neurovascular activity and behavior before, during, and after administration of the treatment in the same subject.

The emitting and measuring steps may occur over more than 5 sleep-wake cycles of said subject.

The method for the detection of neurovascular activity in one or more subjects may comprise:
a) placing each of said one or more subjects into an enclosure in one of the systems according; wherein each of said one or more subjects has said microchip affixed to the skull of said subject for independent measurement of optical signal (e.g., OIS, fluorescence signal);
b) emitting electromagnetic radiation from sources of electromagnetic measurement radiation on the microchip to reflect off of the neural tissue underlying the skull and/or be absorbed by a fluorescent molecule therein of each of said subject; and
c) measuring the reflected light in an optical sensor from each of said microchips;
wherein changes in the reflected electromagnetic radiation correlate with said neurovascular activity. In some embodiments, the method may comprise the simultaneous detection of from one to thirty or one to ten or one to five subjects, where each microchip transmits sensor data to a server (e.g., via microcontrollers in each individual system), and said server aggregates the data collected from each individual microchip. In various implementations, the emitting and measuring steps occur over more than 5 sleep-wake cycles of the subjects.

In particular, a method of monitoring cortical spreading depression (CSD) in a subject (e.g., rat, mouse) expressing channelrhodopsin is provided, wherein said subject expressing channelrhodopsin has a microchip attached to the skull of said subject, and said microchip comprises:
a) a source of electromagnetic measurement radiation arranged on said microchip such that when said microchip is attached to said skull, at least a portion of said electromagnetic radiation from said source reflects off of the neural tissue underlying said skull;
b) an optical sensor arranged on said microchip such that when said microchip is attached to said skull, said optical sensor is configured to senses a portion of said reflected electromagnetic radiation off of the neural tissue underlying said skull; and
c) a source of electromagnetic stimulus radiation; wherein said electromagnetic stimulus radiation is electromagnetic radiation emitted from said source towards said neural tissue underlying the skull; and
wherein the stimulus electromagnetic radiation induces CSD in said subject.
wherein changes in the reflected electromagnetic measurement radiation correlate with the neurovascular activity of the subject; and
wherein the microchip is configured to receive signal from said optical sensor and said microchip is configured to transmit an electrical signal corresponding to at least one property of said electromagnetic radiation;
the method comprising:
a) emitting electromagnetic stimulus radiation from said stimulus radiation to induce CSD in said subject;
b) emitting electromagnetic measurement radiation from said source of electromagnetic measurement radiation; and
c) measuring the reflected electromagnetic measurement radiation with said optical sensor.

In some embodiments, the method further comprises measurement of changes associated with a neurological or systemic disease in a rodent model of such a disease (e.g., rodent models for sleep disorders, Alzheimer's disease, diabetes, or heart disease). The methods may also comprise administering to said subject a candidate substance or device treatment to assess its effects on neurovascular activity, sleep, or behavior or to evaluate said candidate substance for the treatment of said CSD or a disease, disorder, or condition associated therewith (e.g., migraine aura, brain ischemia, seizures). In some embodiments, the method comprises administering to the subject a candidate drug substance or device treatment that that may affect neurovascular activity, sleep, or behavior (e.g., treatments for dementia, depression, sleep disorders, or pain). The emitting and measuring steps may, for example, occur over more than 5 sleep-wake cycles of said subject.

Continuous, minimally invasive recording over extended time periods afforded by the present disclosure reveals patterns of neurovascular activity and behavior that may not be apparent with more invasive techniques. With the microchip-based approach, multiple modalities of recording and stimulation can be combined, with streamlined and synchronized data collection. The OIS sensing microchips of the present disclosure not only reveals cerebral blood volume but, may also be used to quantify heart rate and/or respiration (simultaneously if needed).

Microchips having a movement sensor may provide a highly sensitive readout of head movement, which may afford detection of subtle changes in behavior not visible with video recording techniques. The combination of these recording approaches (e.g., OIS in combination with motion sensors) with an integrated optogenetic stimulation technique enables the quantification of both neurovascular and behavioral responses to triggered brain activity.

These measurements may be performed in freely behaving animals over one or more weeks (e.g., 1 week, 2, weeks, 3 weeks, 4 weeks, 5 weeks, 6 weeks, seven weeks, 8 weeks) and possibly longer for example, up to a year or six months or three months.

There are a variety of other advantages of the approaches described herein. Multiple experiments can be performed in the same subject under different conditions over time, which increases the statistical power of results and thereby reduces the number of subjects required. For pharmacological studies (e.g. methods involving a “candidate substance”), effects from that candidate substance can be examined before, during, and after periods of administration of pharmacological agents, enabling increased quality of data. Since data can be collected and stimulations can be triggered remotely, there is reduced need for handling of mice, minimizing animal stress that may influence experimental outcomes. There is also reduced requirement for in-person monitoring of mice by laboratory personnel, an important issue during the current pandemic. Finally, multiple subjects (e.g., with multiple systems each having their own subject, multiple subjects in one system, multiple subjects in a room such that each is exposed to the same environmental conditions) can be monitored simultaneously, with communication between individual microcomputers and a central server. This server can also receive data from a separate microcomputer that controls and/or monitors room conditions (e.g., light, humidity, barometric pressure). This monitoring of environmental conditions can then be synced with data from individual enclosures. The relatively low expense of the equipment makes it feasible to equip a large number of enclosures, significantly increasing throughput for rodent studies.

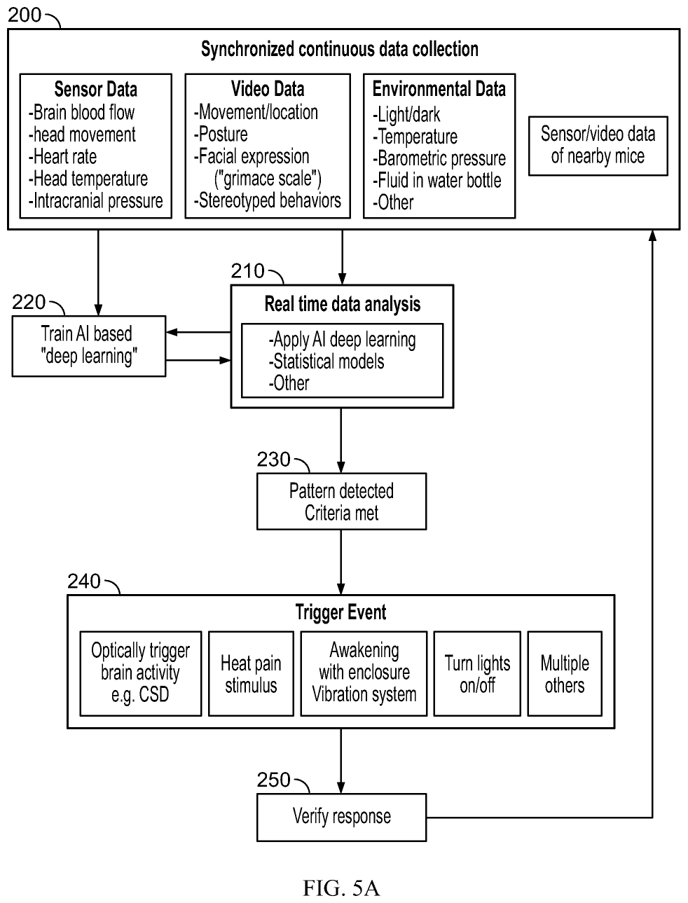

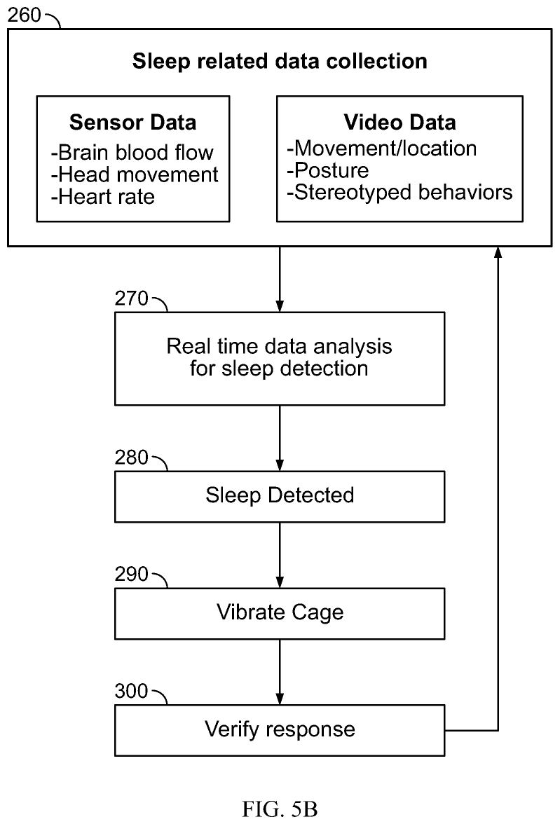

The data collection afforded by the systems of the present disclosure, including synchronized continuous data collection, can be used to elucidate patterns in subjects from measurements as described herein. Those patterns may be used to characterize certain aspects of a subject, such as characterization of a disease state and/or response to a drug candidate substance. Additionally, the systems may trigger various events that stimulate the subject allowing for additional measurements on the physiological/behavioral parameters to be performed. Referring now to FIG. 5A, an exemplary flow chart for software that may perform this analysis (e.g., on one or more servers or microprocessors part of or able to receive information from the systems of the present disclosure that contain storage mediums having instructions to perform these actions). In step 200, data is collected and compiled from one or more measurement systems of the present disclosure. This data may include microchip sensor data such as OIS measurements which may be correlated with brain blood flow or heart rate or other microchip measurements as described herein such as head movement, head temperature, and intracranial pressure. The data may include camera data which may relate to the movement or location of the subject, subject posture, subject facial expression, subject behavior. The data may include data regarding the subject environment during any measurement time point such as room conditions including the amount of light the subject is exposed to during any measurement, the barometric pressure the subject is exposed to during any measurement, the temperature the subject is exposed to during any measurements. Furthermore, the data can be compiled from other subject as well (e.g., within the same room, in separate rooms).

This data or any portion thereof may compiled be transmitted to a server and enable real time data analysis at step 210 by any experimenter using these systems to monitor the subjects. Additionally, data or any portion thereof may also be leveraged machine learning and data processing techniques to identify correlations in the data itself and/or with the data and the real time analysis at step 220. For example, disclosed computerized methods may also allow users to identify trends and collect emergent relationships by aggregating and normalizing user input. The data collected at step 210 may be transmitted to a server configured to interact remotely with one or more devices connected thereto on a computer network such as a local network or the internet. These connections may facilitate monitor, review, and analysis of the data and subject by a user. In some embodiments, the remotely connected user may be able directly or indirectly, via a networked connection, trigger a response in the system such as apply a heat stimulus, stimulate the subject by electromagnetic radiation (e.g., induce CSD), alter the environmental conditions (e.g., light), or deliver a candidate substance.

In certain embodiments, elements for the computer-implemented-system may include a processor configured to perform operations for determining relationships between the sensor data and analysis. This iterative process may be repeated until identifiable patterns emerge from the data such as establishing a baseline state of one or more subjects for measurement comparison. Consequently, at step 230, the system may be able to identify when certain criteria are met and, when criteria are met, event may be triggered in the system at step 240. For example, the brain activity may be triggered with electromagnetic stimulation as described herein, a heat stimulus may be applied as described herein, the enclosure may be vibrated as described herein, or light on the subject may be altered. Measurements (such as those occurring during step 220) may be performed during and after the triggering. The response may be verified at step 250 by the experimenter and/or by the machine learning algorithm by feeding the response data back into the learning loop. This iterative process may be used to develop and train the artificial intelligence algorithms. In various embodiments, the artificial intelligence algorithms may correlate data with various unknown characteristics of a subject such as a response to a drug candidate substance and/or characterization of a disease state. An exemplary loop involving a single subject is provided as FIG. 5B, which shows how the system may maintain an awake state in the subject such as measure parameters at step 260, analyze these parameters at step 270, identify for when subject is asleep (or approaching sleep) at step 270, trigger a response (e.g., vibrate the cage) at step 280, and maintain response until the subject is awake at steps 290 and 300 to ensure the subject maintains an awake state throughout a measurement time period.

A) Heart Rate Monitoring

The OIS, recorded using the microchip technique, includes low-amplitude changes that may be correlated with heart rate. Analysis of these changes enables quantification of heart rate and respiration over extended time periods in conjunction with the CBV and head movement measurements. In addition to acute changes in vital signs associated with different states or experimental conditions, this approach enables long term monitoring of heart rate variability, which has been used as a metric of autonomic function and has been found to be altered in a variety of disease states.

B) Sleep Wake Cycle

Changes in cortical blood flow over the sleep-wake cycle have previously not been extensively characterized, in part because of the inability to record continuously with sufficient time resolution over multiple cycles. However, the systems and methods operative in the present disclosure show that, on average, there is a slow sinusoidal pattern of blood flow change associated with the sleep wake cycle. In general, there is a direct correlation between CBV and head movement and movement within the enclosure. Each time the animal shows sustained movement, an increase in CBV is typically observed, and conversely CBV typically decreases when the animal is at rest. One exception to the correlation between movement and CBV is observed during sleep, when intermittent significant blood flow changes are observed in the absence of any correlated movement. The temporal characteristics and pattern of these changes in CBV are consistent with REM sleep.

The capacity to continuously, noninvasively record multiple sleep-wake cycles such as more than (or up to 90) 10 sleep-wake cycles, more than 20 sleep wake cycles, 28 sleep-wake cycles (or more) in the same animal represents a unique opportunity to quantitatively examine the effects of genetic, environmental, or pharmacological variables on multiple aspects of sleep. Onset and duration of sleep, frequency of awakenings, patterns of REM sleep, and brain blood flow and heart rate during sleep can all be quantified using this approach (e.g., via correlations in motion sensor measurements).

In addition, the effects of pharmacological agents (or “candidate substances”) on the sleep/wake cycle and its individual components can be investigated for multiple sleep-wake cycles such as from 2 to 90 sleep-wake cycles. The effects can be accurately determined by quantifying brain neurovascular activity and behavior during the sleep/wake cycle before, during, and after administration of the candidate substance.

C) Minimally Invasive Triggering and Recording of CSD

CSD is a multiphasic event that begins with a slowly propagated wave of depolarization of neural tissue, followed by a prolonged inhibition lasting 60 minutes or longer. These electrophysiological changes are accompanied by profound changes in blood flow and uncoupling of the normal vascular response to neural activity. CSD is believed to be the physiological substrate of the migraine aura, and also occurs in the setting of brain injury. It has been used for decades as a translational model of migraine and is believed to be a therapeutic target in brain injury.

The majority of studies of CSD in rodents have been performed under anesthesia, and those that have been performed in un-anesthetized animals have involved significantly more invasive methods than those described in the current study. The present disclosure offers the ability to trigger and record CSD repeatedly in a freely behaving animal over extended time periods represents a new opportunity to examine the effects of CSD on physiological and behavioral parameters, in addition to examining the effects of genetic, hormonal, circadian factors on CSD susceptibility and its clinical characteristics (or combinations thereof).

In addition, the effects of pharmacological agents (or “candidate substances”) on CSD and its behavioral consequences can be investigated for both individual CSD events as well as for repeated CSD events over extended time periods.

D) Behavior

The combination of a sensor for head movement with a method for real-time tracking of position within an enclosure enables new approaches for investigating behavior. For example, it is not only possible to determine if the animal moves in general with the systems of the present disclosure, but also objectively determine if a subject moves its head in a specific way, such as with a “wet dog shake” or a tottering pattern that may be linked to specific pathologies such as pain, epilepsy, ataxia, and multiple others. There is also the possibility of identifying other subtle patterns of head movement associated with specific behaviors as measured from one or more motion sensors on the microchip. The ability to monitor movement within an enclosure in real time may create the possibility to deliver specific positive or negative stimuli based upon location, facilitating conditioned place preference or conditioned place aversion studies.

E) Estrous Cycle Measurements

The microchip systems of the present disclosure may be used to track the estrous cycles of subjects. These measurements may be performed by microchip systems comprising one or more thermal sensing microchips affixed to the skull. The thermal measurements of the skull surface temperature may be correlated with the estrous cycle by, for example, vaginal cytology measurements of the subject over the course of one or more estrous cycles. In estrous cycles, these measurements may then be correlated to other patterns identified from the other sensors, such as OIS measurement sensors, ICP, and motion.

### EXAMPLES

The following examples illustrate specific aspects of the instant description. The examples should not be construed as limiting, as the example merely provides specific understanding and practice of the embodiments and its various aspects.

Methods

All studies were performed according to IACUC guidelines and requirements and were approved by the UCLA Animal Research Committee.

Mice

C57Bl6 male and female mice, aged 14-25 weeks, weight 22-28 grams were used for some studies. For studies in which CSD was optically triggered by activation of channelrhodopsin (ChR), two- to six-month-old homozygous male and female transgenic mice that expressed ChR2-eYFP fusion protein in layer 5 cortical neurons were used (Strain 7612—B6. Cg-Tg(Thy1-COP4/EYFP)18Gfng/J; Thy1/ChR2; the Jackson Laboratory, Bar Harbor, ME, USA). All animals (mice and rats) were kept in standard grouped housing (3-4 animals per cage) except during measurements when the animals were housed individually. Room conditions were kept at 20-23° C. and 30-70% humidity with a normal 24 hr dark/light cycle (12 hours on, 12 hours off). All animals were fed standard rodent food pellets (Teklad laboratory diet, rodent diet, Envigo, Madison, WI).

Cortical spreading depression (CSD) in freely behaving mice was triggered optically by administering sequential pulses of light with increasing duration until CSD was observed based on OIS. After each light pulse, OIS was monitored for at least 10 minutes for the occurrence of CSD. If CSD occurred, no further stimulus pulses were delivered, whereas if no CSD occurred, then the duration was increased with the subsequent pulse. To trigger CSD under anesthesia, the cage that the animal was housed in was rapidly flushed with 2% isoflurane in a 20/80% O₂/N₂ gas mixture. After the animal lost consciousness, the isoflurane was reduced to 1.5% to sustain anesthesia. For prolonged experiments a heating pad set at 37° C. was placed under the cage and a small paper blanket was placed over the animal to maintain its core temperature. Breathing pattern and paw color were continuously monitored. Anesthesia was maintained until CBV returned to baseline levels at the end of the CSD.

Rats

Sprague Dawley male rats, postnatal day 25-35, weighing 100-150 grams were used for some OIS recording studies.

Microchip and Video System

For long term continuous recordings in freely behaving animals, microchips were permanently affixed to the skull in a short (<30 min) surgical procedure. Briefly, anesthesia was induced with 5% isoflurane, which was immediately lowered to ˜2% for the placement into the stereotaxic frame and the surgery. Throughout the rest of the surgery, isoflurane was maintained at ˜1.5%.

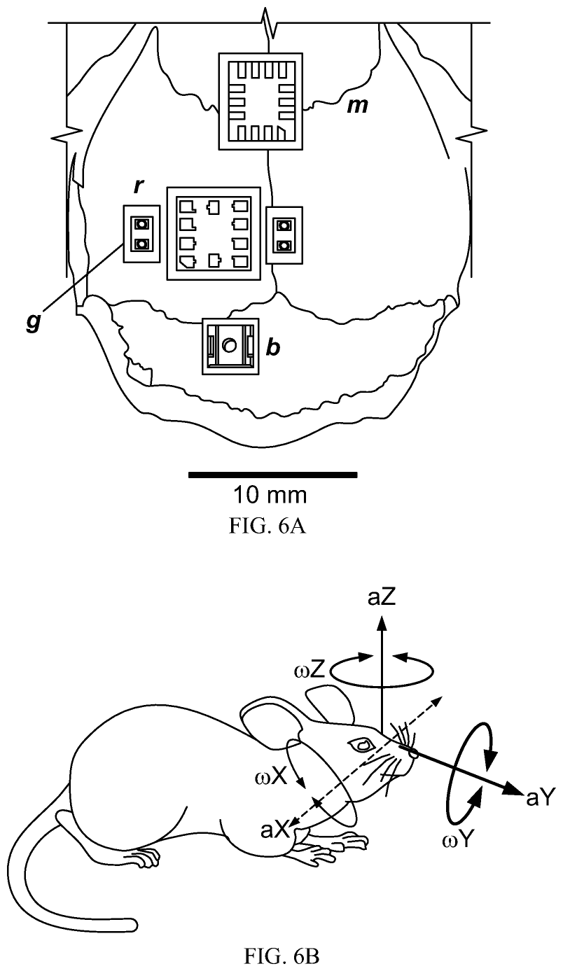

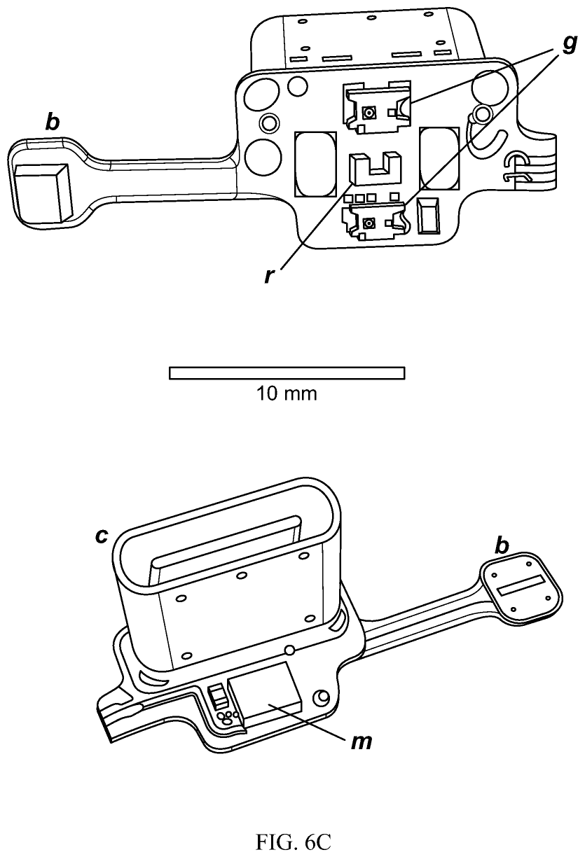

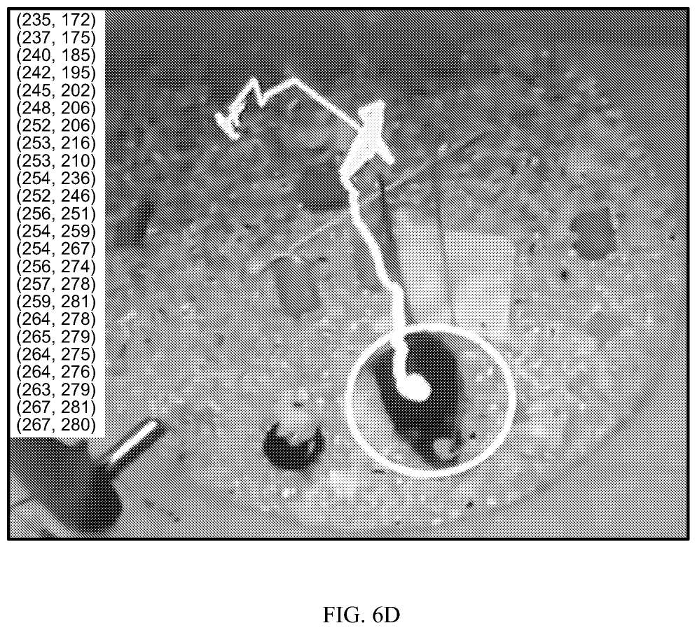

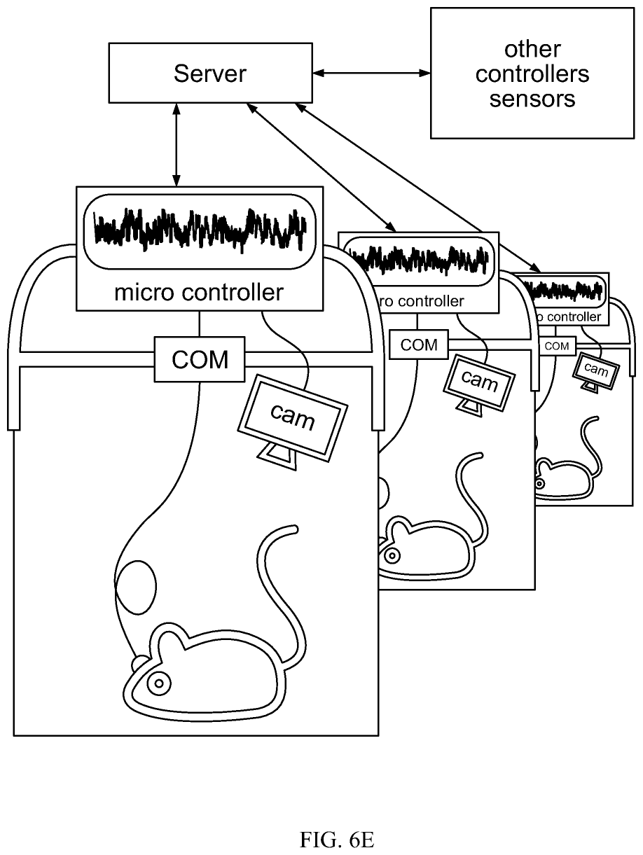

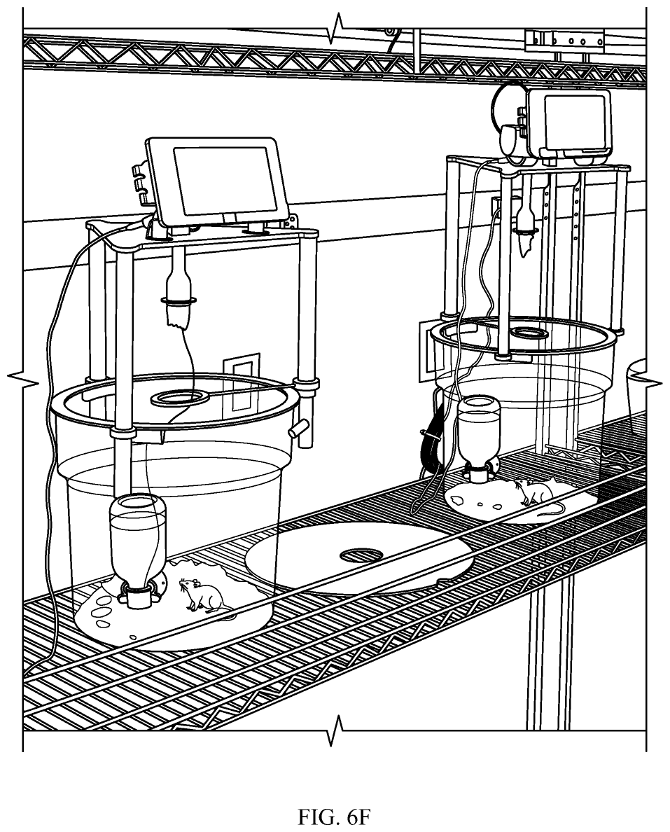

The skull was surgically exposed, and the chips were glued onto the skull with cyanoacrylate and permanently enclosed with black two-component resin (J-B Kwik, J-B Weld Company, TX, USA). The chip system was comprised of an optical sensor to measure reflection, green LEDs for reflectance/OIS illumination, a blue LED for optical stimulation of ChR, and a movement sensor (FIG. 6A). The motion sensor records acceleration in three directions (aX, aY, aZ) as well as angular momentum (ωX, ωY, ωZ) (FIG. 6B). All chips were embedded on a small circuit board which also contained a waterproof connector so that the animals could be disconnected and reconnected if needed (FIG. 6C). The custom designed flex-print circuit boards were professionally manufactured in small batches (Syrinx Industrial Electronics, Nibbixwoud, The Netherlands).

In addition to the chip recordings, the system also recorded a video which is linked to a real-time tracking system that continuously tracks the location of the animal with a short delay (<50 ms) (FIG. 6D). The tracking system uses an opensource computer vision framework to detect the mouse on the video frame and determine its x and y coordinates (FIG. 6D). To achieve maximal detection speed, a simple object detection model from the Open Source Computer Vision Library (OpenCV 3.2.0, www.opencv.org) was used. This detection model searches for objects with a pre-defined color range. The model is capable of reliably detecting dark mice in contrast with light bedding. The accuracy of the model was verified visually by plotting a circle around the detected mouse on the video frame and printing the detected coordinates. In FIG. 6D, measured coordinates can be seen on the left side of the image with an exemplary track for the subject under measurement.

During subject monitoring, the microchip system was connected via recording wires to a micro-controller/computer unit (FIG. 6E, Raspberry Pi 3b or 4b). A custom-designed and 3D-printed commutator ensured range of movement of the wires throughout the enclosure without tangling. The video camera was also connected to the microcontroller for recording video signals. The microcontroller unit controlled all components of the microchip system, collects data from the system, and records video. The microcontroller unit can also be programmed to do real-time analysis, e.g. to detect specific patterns in the OIS or to determine the subject's location and directly respond to the measured parameters.

Multiple systems were connected to a server for data backup and also functioned as a central control for each individual system. Moreover, the server controls and was configured to measure other, more general conditions, such as room lighting and climate controls. Each enclosure in the systems had a custom-designed and 3D-printed superstructure, upon which the commutator, camera, and microcontroller with a touch screen were mounted. (FIG. 6F).

Data Analysis

All software for the Raspberry Pi used in the experiments performed on the systems described herein was written in Python 3 (www.python.org). Data were analyzed using Python, NumPy (www.numpy.org), SciPy (www.SciPy.org), and Igor (Wavemetrics, Lake Oswago, OR). CSD under anesthesia and awake were compared by duration and amplitude and area under the curve (AUC). A baseline value was established over 5 minutes before stimulation. Area under the curve was calculated over a window of 2 h from the start of the CSD. P values were calculated using the students T-test.

OIS data measured with the microchip in the acute experiments were sampled at 1024 Hz, while OIS and movement data in the long-term experiments were sampled with 128-256 Hz. Optical sensors detected changes in light intensity.

Validation of Microchip Based OIS Measurements

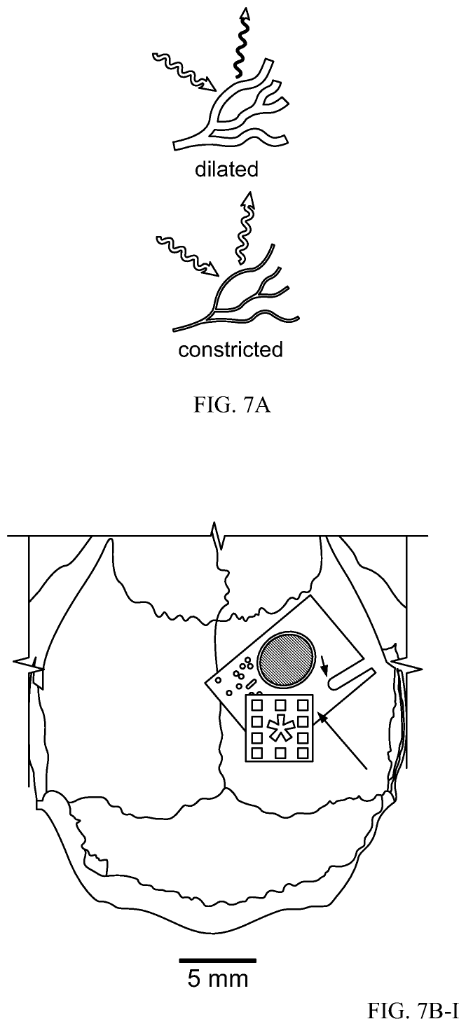

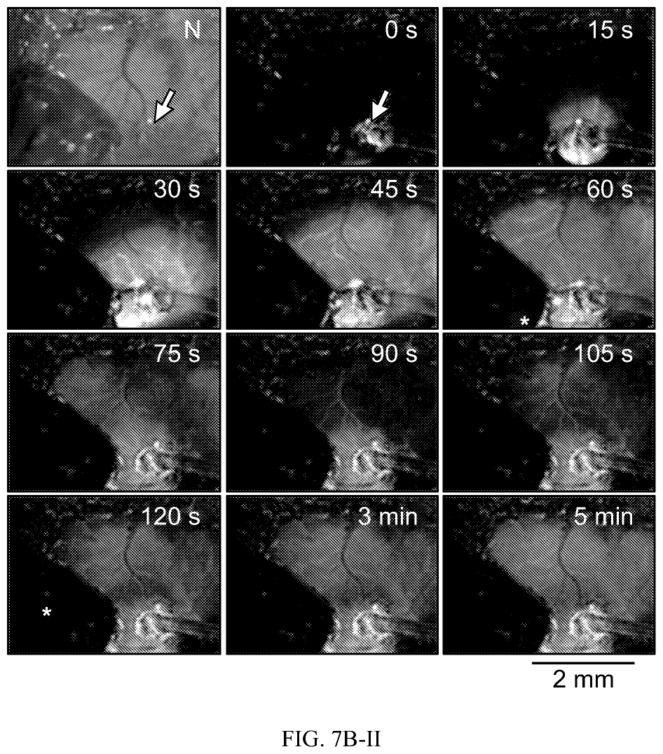

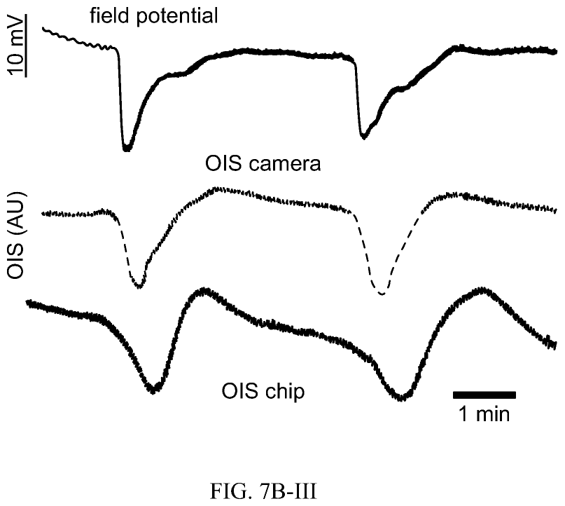

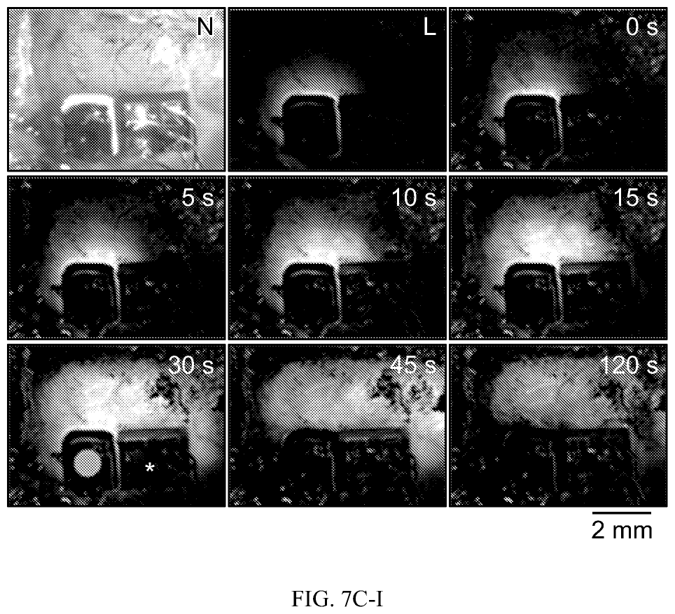

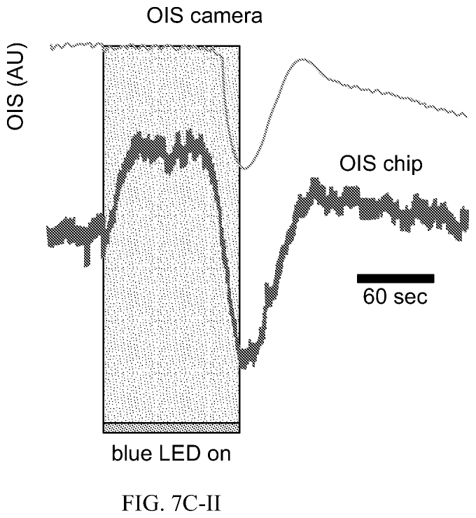

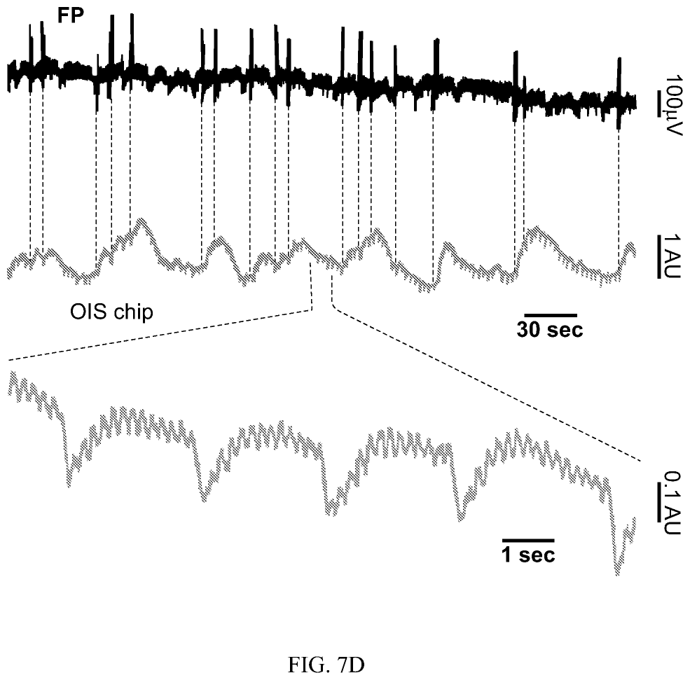

Experiments validating microchip triggering and recording were performed with Thy1ChR2 mice and compared to those described in Pradhan A A et al, Br J Pharmacol 171 (2014): 2375-2384, which is hereby incorporated by reference in its entirety and particularly in relation to the OIS measurements performed therein. Briefly, animals were anesthetized and placed into a stereotaxic frame. During recordings of CSD, isoflurane was maintained at ˜1.5%. After induction of anesthesia, the skin from the skull was detached and a rectangular region of ˜2.5×3.3 mm 2 (˜0.5 mm from sagittal and ˜1.4 from coronal and lambdoid sutures) of the right parietal bone was thinned to transparency with a micro drill. The brain was illuminated (through the translucent window) with a green LED (565-575 nm). Images were captured with a resolution of 1040×1392 at 1 Hz with a High-Sensitivity USB Monochrome CCD Camera (Mightex Systems, Pleasanton, CA). A schematic of the OIS measurements illustrate reduced reflectance of green light when vessels are dilated and there is increased blood volume is shown in FIG. 7A.

Throughout the experiments and data described herein, OIS is expressed relative to baseline OIS determined as the 24-hour rolling median of the OIS. Downward changes in OIS traces indicate a decrease in CBV and vasoconstriction (increased reflectance) whereas upward changes indicate an increase in CBV and vasodilation.

The optical sensor component of the microchip system was glued to the skull immediately adjacent to the thinned skull window used for camera imaging (FIG. 7B-I). A small burr hole was drilled through the skull adjacent to the window for placement of an electrode for field potential recording. A second small burr hole was drilled through the skull adjacent to the window, into which a tube was inserted. CSD was triggered by application of a drop of 1 M KCl applied through this tube. Alternatively, CSD was triggered by blue light stimulation from an ultrabright blue LED (460 nm, FIG. 7C-I grey dot in 30s image). For the un-anesthetized in-vivo experiments the same LEDs for green light (2-4 LEDs, total light optical output set to 5-10 μW) and blue light (optical output set to 25 mW) were used. Independent experiments showed that the LED's temperature increase was negligible (<1° C.).

In all plots OIS is expressed as a change in OIS compared to baseline OIS determined as the 24-hour rolling median of the OIS. The assumption is that over a full day cycle the animal will have an even median OIS, i.e. the animal has comparable conditions and duration over 24-hour blocks for factors that could influence OIS, such as sleep or feeding patterns. OIS changes are plotted so that corresponding increase in CBV (cortical blood volume) and vessel diameter are upward (i.e. a reflection decrease in OIS is plotted upward, while a reflection increase is plotted downward).

### Example 1: Validation of Microchip-Based Recording Method

OIS is the passive reflection of light from tissue which can change due to the biophysical properties of the tissue. In our approach we illuminated the cortex through the skull with green light which is primarily absorbed by blood such that at this wavelength, OIS is correlated with blood volume in the tissue. The blood volume is in turn correlated with the dilation (increased absorption or darkening of the tissue) or constriction of blood vessels (increased absorption or lightening of the tissue (FIG. 7A).

To verify the ability of the microchip components to record OIS and to trigger CSD, several acute experiments were performed on anesthetized animals described above. In these experiments OIS recorded with the microchip was compared to simultaneous video recordings of OIS performed with a camera through a window of thinned skull (FIG. 7B (FIGS. 7B-I, 7B-II, 7B-III) and 7C (FIGS. 7C-I and 7C-II).

In one set of acute experiments (n=2) CSD was triggered with a drop of 1M KCl directly placed on the cortex through a hole in the skull (FIG. 7B-I). This resulted in changes in OIS recorded with the optical sensor of the microchip system that were remarkably similar to those recorded with the camera and were correlated with the caliber of cortical surface arteries visualized with video (FIGS. 7B-I and 7B-II). These changes paralleled depolarizations observed with extracellular field potential recordings (FIG. 7B-III). In another experiment (n=1) we used a blue LED glued to the skull to trigger CSD in animals that express ChR in cortical pyramidal cells. After a short illumination (<1 min) with the blue light a CSD was triggered that was similar to that observed when CSD was triggered by KCl and could easily be observed with either the camera or the OIS recording microchip. (FIG. 7C).

### Example 2: Neurovascular Coupling, Breathing and Heartbeat

In addition to detecting large signals such as those that occur with CSD, the optical sensor component of the microchip system is capable of detecting smaller changes in blood volume that result from normal neurovascular coupling. Deep anesthesia results in repetitive bursts of cortical action potentials with intervening silent periods also known as burst suppression activity.

Simultaneous field potential recording and video recording showed that repetitive cortical bursts are associated with repetitive dilation of blood vessels. Similar changes are observed with OIS recorded using the microchip optical sensor. Each burst of action potentials was associated with a change in OIS consistent with an increase in cerebral blood volume due to normal neurovascular coupling (FIG. 7D).

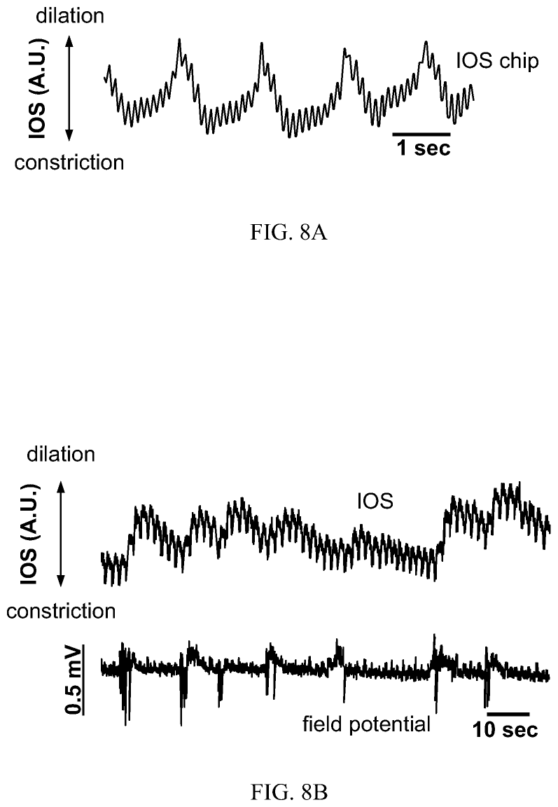

One of the other major advantages of measuring OIS with a microchip compared to a camera is that the microchip can continuously sample at much higher rate than a camera. In the acute experiments, the microchips sampled 1 kHz and are able to detect much faster changes in blood volume than compared to typical measurement techniques. Increased resolution from the OIS from the microchip reveals oscillations with 10-50× smaller amplitude than the changes in signal associated with neurovascular coupling. One of these is an oscillation of 0.6-1 Hz consistent with the breathing of the animal. Superimposed on this breathing oscillation is an even smaller amplitude OIS oscillation with a frequency of 7-12 Hz consistent with the arterial pulse. This small and fast OIS oscillation indicative of the heart rate was not be detected by the camera, which samples at a significantly lower OIS measurement frequency (1-2 Hz). Higher resolution traces of the OIS illustrating these as measured from the microchip can be seen in FIGS. 8A and 8B. In FIG. 8B, a simultaneous electrophysiological recording was taken on the mouse illustrating that increases in blood flow correspond with brain activity (i.e., neurovascular coupling is evidenced).

### Example 3: Continuous Monitoring of Cortical Blood Volume (CBV) and Movement in Freely Moving Rodents

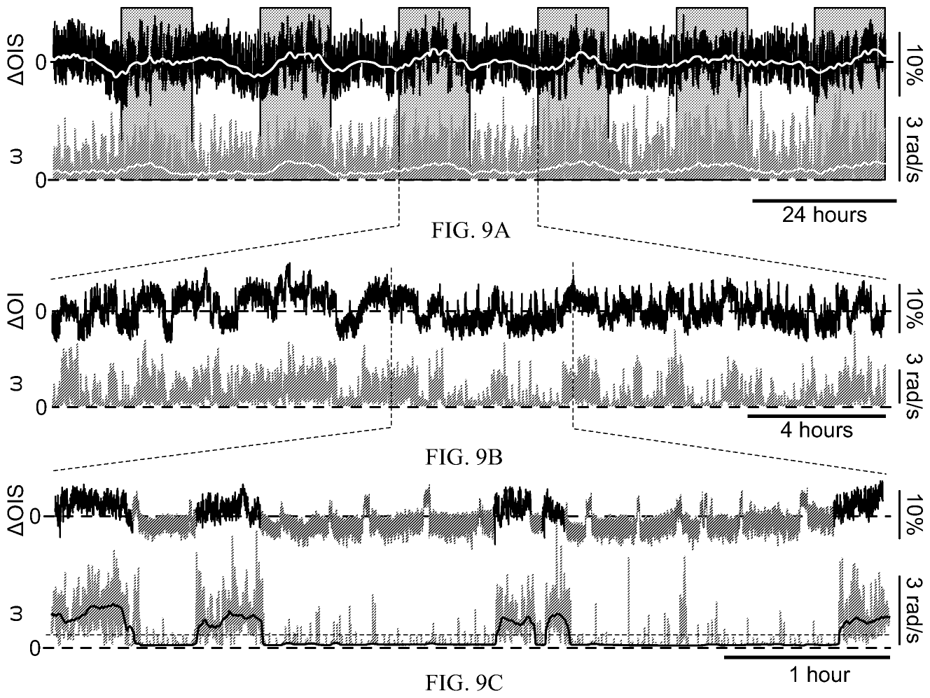

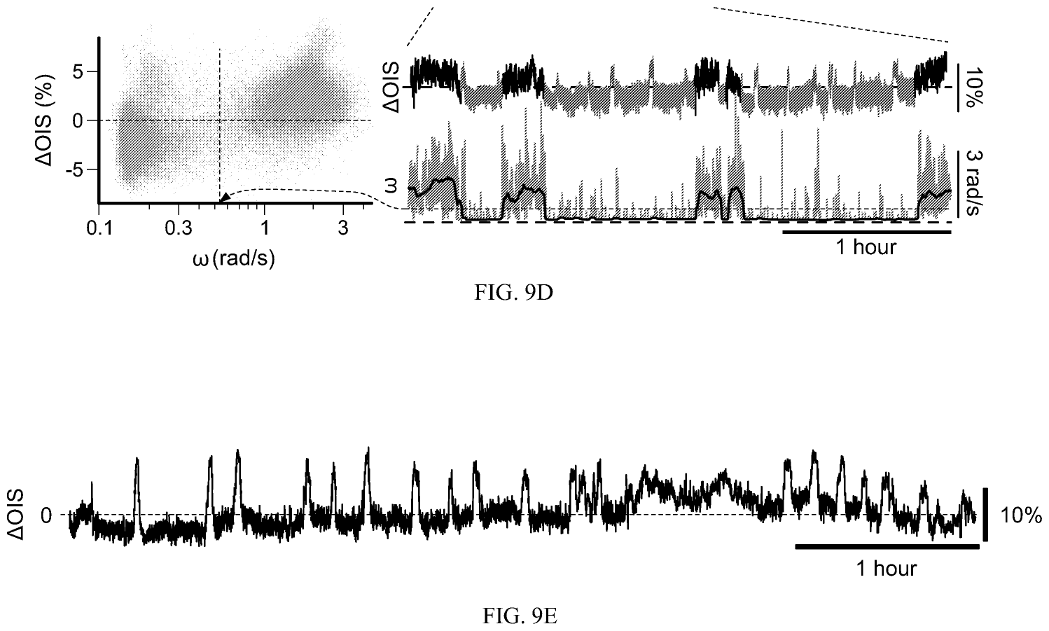

When microchips of the present disclosure are permanently affixed to the skull, the system enables continuous recording of high-quality data in freely behaving mice (n=6, FIGS. 9A-E). Intermittent recordings were made up to 100 days after attachment of the microchips, with continuous recordings lasting up to 28 days. All experiments were terminated (17-100 days after attachment) for reasons other than experimental complications (e.g. due to COVID-19 shutdown of research activity) and could have lasted longer.

Long-term recordings over multiple days show a 24-hour pattern of fluctuation in CBV that corresponds with increased and decreased movement over the day/night cycle (FIG. 9A).

Examination of time intervals with higher resolution shows that periods of head movement are consistently associated with periods of increased CBV (FIG. 9B). Conversely, there are sustained periods of decreased movement and decreased CBV (FIG. 9C). Examination of video recordings confirmed that these prolonged inactive periods were consistent with sleep. When plotting OIS against movement, two clouds become evident indicating at least two distinct states of movement, one correlated with sleep and one with wakefulness (FIG. 9D (left)). Since the two distributions of movements are quite distinct, the movement can easily be used to automatically detect periods of sleep (FIGS. 9C and 9D (right)). Remarkably, during the sleep period, there are consistently transient large increases in CBV that do not correspond with increases in movement. These increases have temporal characteristics consistent with REM sleep in mice (Daszuta et al., Brain Res 283 (1983): 87-96; Perez-Atencio et al., PLos One 13 (2018): e0189931; Turner et al., Elife 9 (2020), each of which are hereby incorporated by reference in their entirety) and the increases in CBV are consistent with increases in blood flow that have been reported during REM sleep in humans (Hajak et al. Sleep 17 (1994): 11-19, which is hereby incorporated by reference in its entirety). Similar OIS recordings have been recorded for up to 4 days in rats (n=3, FIG. 9D), indicating that the thickness of the rat's skull does not compromise OIS recording with microchips.

Heart Rate

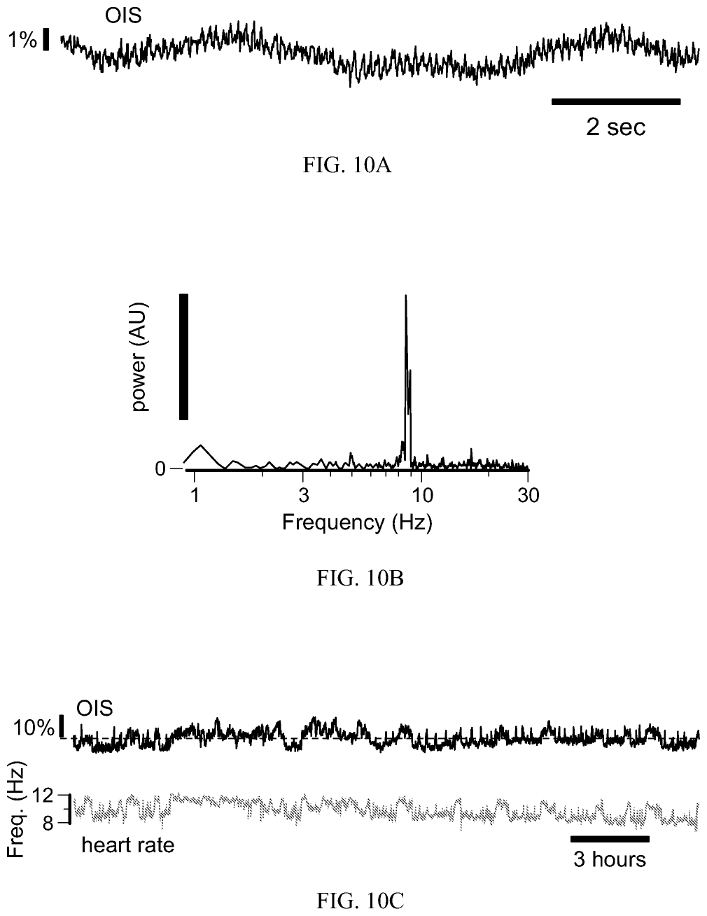

The OIS contains a high frequency, low amplitude component that is consistent with the arterial pulse (FIGS. 7D, 10A) seen only in OIS measurements performed with the presently disclosed microchips, systems, and methods. Using the techniques described herein, this signal component could easily be detected in the fast Fourier transform of 10-second sections of OIS recording (FIG. 10B) and can be used to continuously track the heart rate of the animal. This analysis shows that the heart rate is generally correlated with activity, increasing during the wake state and decreasing during the sleep state (FIG. 10C). There is a bimodal distribution of heart rate corresponding with the wake and sleep states (not shown).

Cortical Spreading Depression (CSD)

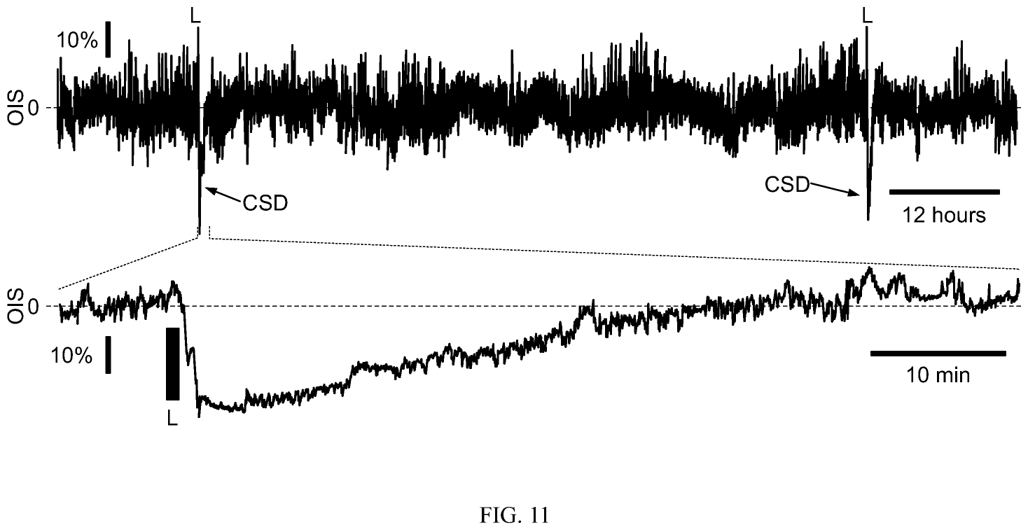

CSD can be consistently triggered and recorded in freely behaving mice using the microchip systems of the present disclosure (FIG. 11). The duration of light required to trigger CSD can be used as an indication of CSD threshold. The OIS changes observed are similar to those observed with a video camera, including an initial transient increase in OIS consistent with a decrease in CBV that occurs with the propagated wave of depolarization (Phase 1). This is followed by a prolonged global decrease in CBV lasting 30-60 minutes (Phase 2) (FIG. 11). We have successfully triggered consistent CSD events multiple times per day (n=49 in 5 animals), and on multiple days in a row over a 28-day period.

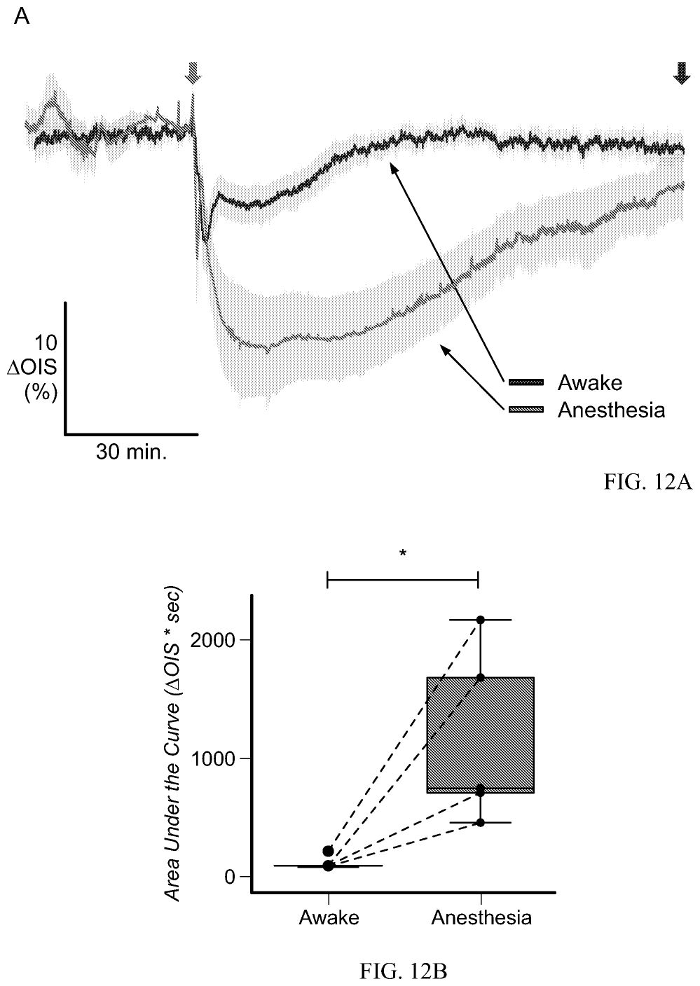

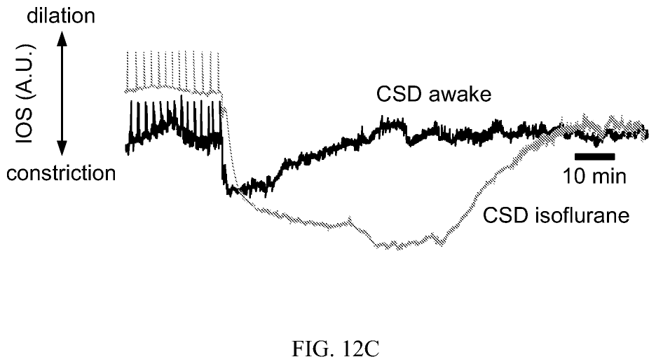

Examination of CSD in freely moving animals in the awake state vs. under anesthesia showed marked differences in the amplitude, duration, and other kinetics of changes in CBV (FIGS. 12A and 12C). As an example of these differences, quantification of the overall amplitude and duration of the decrease in CBV as area under the curve (AUC) reveals that there was approximately 10 times less net vasoconstriction when the animal was awake (112.27 ΔOIS*sec.) vs. under anesthesia (1148.92 ΔOIS*sec.; n=5; p=0.03).

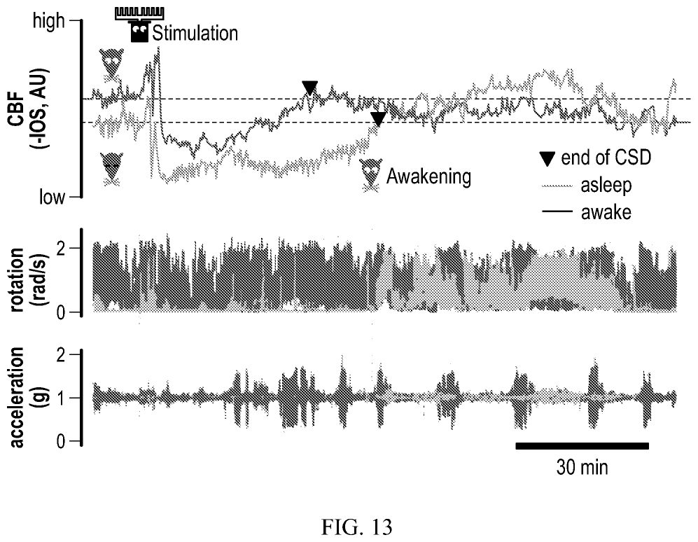

Detailed traces of the cerebral blood flow (CBF) as measured from IOS chips and correlated with sensor measurements is shown in FIG. 13. As can be seen, CSD provides different responses when triggered in the awake state as compared to the sleep state. These results have significant implications regarding the interpretation of CSD results as a translational model.

The importance of studying neurovascular phenomena like CSD in freely moving animals is clearly illustrated by present examples showing the marked differences in the characteristics of CSD under anesthesia as compared with the awake state. In addition to implications regarding basic physiological mechanisms in different brain states, these findings have potential clinical relevance. Clearly most migraine does not occur under anesthesia, whereas in the setting of significant brain injury, sedation or anesthesia may in fact be employed as part of clinical management, and therefore may affect the CSD and its potential role in patient outcomes.

### Example 4: OIS Microchips Having Pressure Sensors

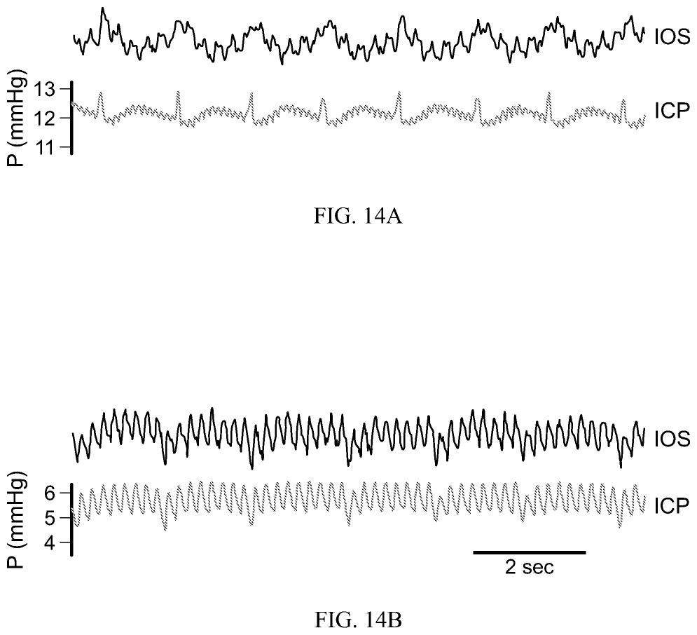

A pressure sensor capable of measuring intracranial pressure (ICP) with a resolution of 6 mPa and a sample rate of ≥70 Hz was placed on OIS microchips to allow for simultaneous measurements and OIS. Microchips were affixed to the skull of subjects and simultaneous measurements were performed of OIS and ICP. The simultaneous OIS and ICP measurements taken on a mouse are shown in FIG. 14A and those on a rat are shown in FIG. 14B. As can be seen, particularly in the mouse traces, increases in intracranial pressure correlate with increases in CBF dilation (as measured by OIS).

### Example 5: OIS Microchips Having a Heat Stimulus

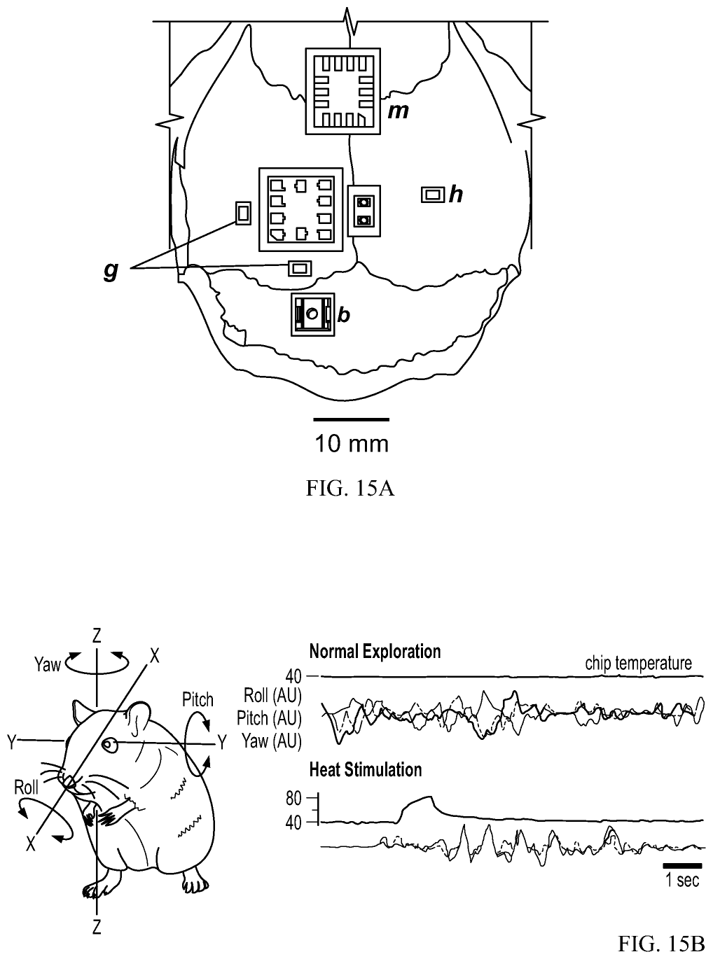

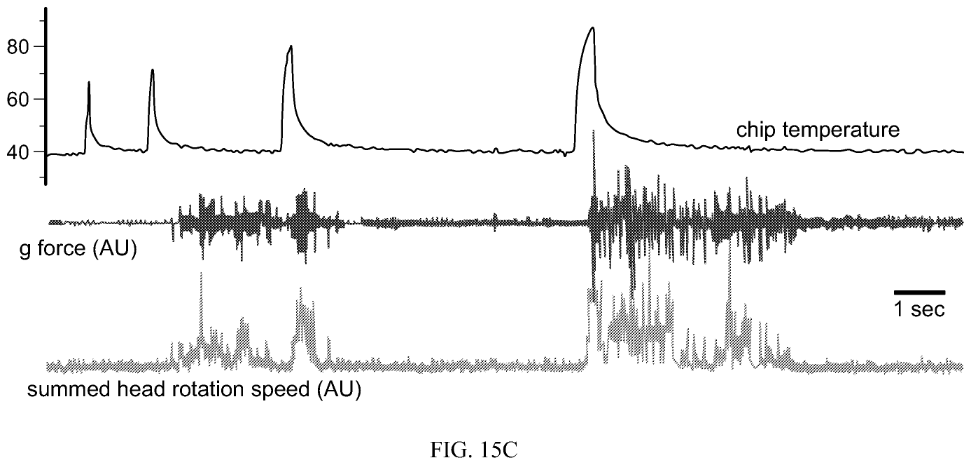

A heat stimulus that generates a temperature wave spike from room temperature to a high temperature (based on current applied across a resistive heater that can approach 120° C.) was placed on an OIS measuring microchip and affixed to a mouse skull. The microchip also included a motion sensor configured to independently measure motion in the roll, pitch, and yaw of the skull of the subject. A schematic of the chip is illustrated in FIG. 15A as positioned on the skull. The schematic illustrates the relative positioning of each component of the chip on the skull to illustrate the source electromagnetic measurement radiation (g), the optical sensor (r), the source of electromagnetic stimulus radiation (b), and the heat stimulus (h).

A comparison of the motion sensor measurements on the roll, pitch, and yaw taken during normal exploration and those during heat stimulation is shown in FIG. 15B (also illustrating a reference frame for these measurements). The temperature stimulus is also shown. As can be seen, the heat stimulus triggers characteristic head movement as compared to normal movement. FIG. 15C illustrates the g force due to head acceleration and the summed head rotation from these components in relation to the heat stimulus occurring with increased temperature and duration. These altered heat stimuli create variations in subject response (as measured the motion sensor). This technique can be used to test sensory thresholds, and to valuate pain responses (e.g., from CSD). Additionally, these thresholds and responses can be compared and potentially correlated with OIS measurements performed simultaneously with the microchip and system.

### Example 6: Heart Rate from the OIS Signal

To confirm high frequency signals in the OIS measurements represent heart rate, simultaneous OIS-ECG recordings were performed on a mouse under anaesthesia in two acute experiments. First, an electrocardiogram (ECG) was measured with two needle-electrodes subcutaneously placed at each armpit of the mouse. AC signal was amplified (100×) and filtered (LP 500 Hz) with an A-M systems model 3000 amplifier (Sequim, WA, USA). Second, OIS recordings were compared with ECG monitor/ultrasound combination: the Visual Sonics Vevo 3100 (FUJIFILM Visual Sonics, Canada) paired with the Scintica Rodent Surgical Monitor+ (Scinta Instrumentation, London, Canada). Another mouse was implanted with an OIS microchip system of the present disclosure that sampled at 1 kHz which was mounted under anaesthesia on the surgical platform (7 days after system implantation). Conductive gel (SignaGel, Parker Laboratories Inc., Fairfield, NJ) was applied to the paws which were taped to the electrodes on the platform. OIS and ECG were recorded simultaneously.

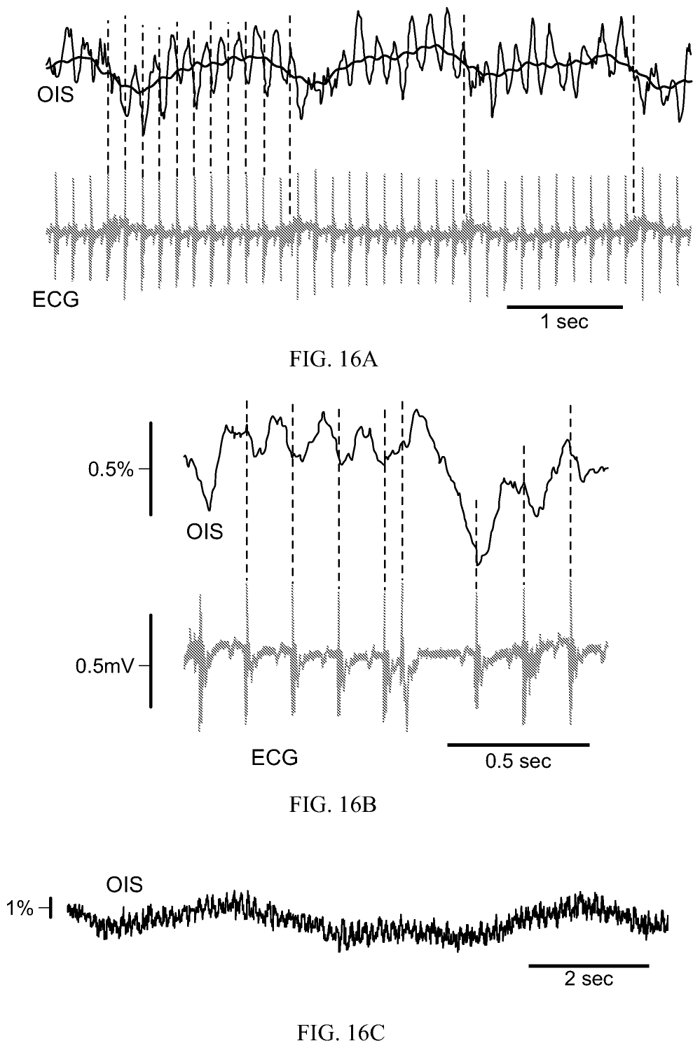

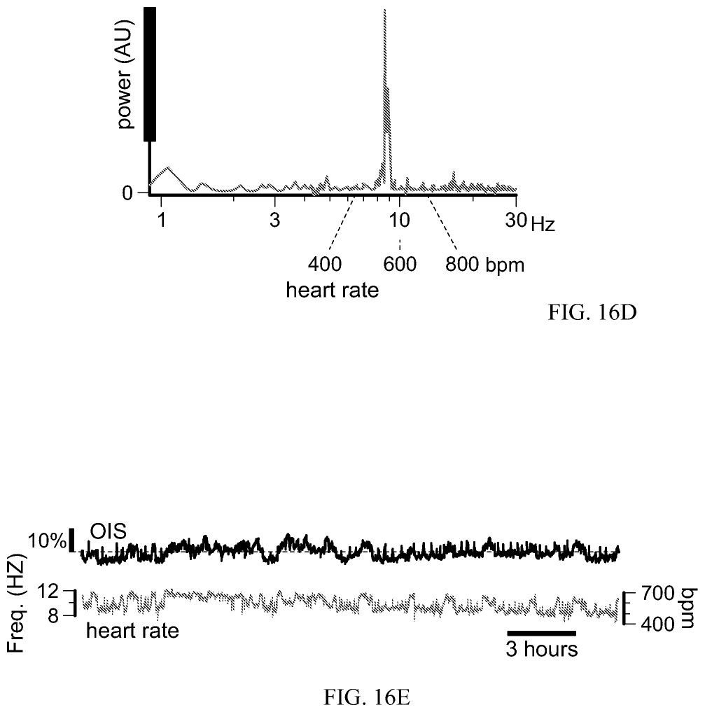

In comparing the OIS/ECG measurements, one of the major advantages of measuring OIS with a microchip system as compared to typical measurement protocols is seen. The OIS systems of the present disclosure can continuously sample activity at a much higher rate than a camera. Cameras capable of performing OIS measurements do not have the measurement frequencies associated with these devices and cameras with measurement frequencies comparable to the present systems are impractical for OIS use due to, for example, increased measurement complexity, lower limits of detection, and expense. Evaluation of the microchip OIS reveals oscillations with 10-50× smaller amplitude than changes in signal associated with cortical burst activity. Of these, an oscillation of 0.8-1.5 Hz correlated with the breathing of the animal. This oscillation is consistent with the respiratory rate that has been previously reported by Ewald et al., Cold Spring Harb Protoc 2011, pdb.prot5563, and Tsukamoto et al., Exp Anim 64 (2014) 57-64, each of which is hereby incorporated by reference in their entirety. The correlation between the OIS oscillation at this frequency and respiratory rate was confirmed by simultaneous measurement with a commercially available small animal heart rate and breathing monitoring system (Visual Sonics Vevo 3100 paired with a Scintica Rodent Surgival Monitor+System). Moreover, when combining OIS measurements with ECG measurements, small breathing artefacts in the ECG line up with a slow wave detected in the OIS (FIG. 16A) supporting the confirmation that breathing underlies a slow oscillation of the OIS.

Superimposed on this respiratory rate oscillation is an even smaller amplitude OIS oscillation with a frequency of 420-720/min (7-12 Hz) consistent with the arterial pulse (FIGS. 7D and 16A-E). This small and fast OIS oscillation indicative of the heart rate would not be detected in camera based measurements, sampling at significantly lower frequencies.

Simultaneous ECG recording with OIS microchip system shows a 1:1 correlation of QRS complexes measured with an ECG and low-amplitude OIS oscilations (with some variability in the timing of the OIS oscillations relative to the QRS complexes as seen in FIG. 16A). A clear confirmation of the correlation between OIS and heart rate is shown in FIG. 16B, in which a premature heartbeat and subsequent delay in the following beat causes a correlated delay in the subsequent OIS peak. Additionally, the premature heartbeat caused a slight decrease in the cortical blood volume (CBV) during the delay. When comparing OIS measurements with a heart rate monitoring system, the measured heart rate was identical, further supporting that the fast oscillations measured in the OIS are due to the heartbeat.

Under unanesthetized conditions, the low amplitude heart rate signal in the OIS is still present as can be seen in FIG. 16C where heart rate can be detected by frequency analysis.

Fast Fourier transforms were taken from sequential 10 s epochs of the OIS signal. To determine the HR, the power in a frequency window which contained the expected the HR (5-20 Hz or 300-1200 bpm) and always found a large peak within a narrow frequency band. The location of this peak was taken as the HR. This heart rate signal component could be detected in the fast Fourier transform of 10-s sections of the microchip OIS recording (FIG. 16D) and can be used to continuously track the heartrate of the animal.

This analysis illustrates that the heart rate is generally correlated with activity, increasing during the awake state and decreasing during the sleep state (FIG. 16E). There is a bimodal distribution of heart rate corresponding with the awake and sleep states. The OIS signal analysis enables quantification of heart rate and respiration over extended time periods in conjunction with the CBV and head movement measurements. In addition to acute changes in vital signs associated with different states or experimental conditions, this approach enables long term monitoring of arterial pulse variability. Although there is a 1:1 relationship between QRS complexes measured on ECG and peaks/troughs of low-amplitude oscillations in OIS, the timing of the OIS oscillations relative to the QRS complexes may vary slightly.

Without wishing to be bound by theory, this variance may be because of other factors, such as baseline blood pressure, vascular resistance, and capacitance. While heart rate variability (HRV) may contribute to variability in the OIS oscillations, they are not identical. Since HRV has been used as a metric of autonomic function, and since autonomic function has been altered in a variety of disease states, OIS oscillations may provide information regarding autonomic function and disease states similar to that provided by HRV.

### Example 7: Different Characteristics of Cortical Spreading Depression in the Sleep and Wake States

Homozygous C57BI6 male and female transgenic mice, aged 8-24, weight 22-28 grams, expressing ChR2-eYFPfusion protein in layer 5 cortical neurons were used (Strain 7612-B6.Cg-Tg(Thy1-COP4/EYFP) 18Gfng/J; the Jackson Laboratory, Bar Harbor, ME, USA). Mice were kept in standard grouped housing whereas during measurements the animals were housed individually. A normal 24-hour dark/light cycle (12 hours on/12 hours off) was maintained. Following the experiments, mice were euthanized by cervical dislocation under isoflurane anesthesia.

Microchips as described herein and in Yousef Yengej, et al, J Physiol. 599 (2021): 4545-4559, which is hereby incorporated by reference in its entirety particularly in relation to its microchip systems. Carpofen (5 mg/kg s.c.) and topical 2% lidocaine were administered as analgesics at the time of the procedure. Experimental data collection started >3 days after microchip attachment. The microchip system (Syrinx Industrial Electronics, The Netherlands) was comprised of an optical sensor and green LEDs for illumination and OIS measurements, a blue LED for optical stimulation of channelrhodopsin, and a movement sensor. Implants were connected via recording wires to a microcomputer (Raspberry Pi Foundation, Cambridge, UK), running software as described in YousefYengej, et al, J Physiol. 599 (2021): 4545-4559, which is hereby incorporated by reference in its entirety, particularly in relation to the software used. Heart rate was isolated from OIS using spectral analysis and Fourier Transforms taken over a 10-second rolling time frame. Heart rate was determined by the power in a frequency window which contained a heart range within an expected range (5-20 Hz/300-1200 bpm).

CSD was triggered with optically sequential light pulses (blue LED) with increasing duration (1-10, 10-20, and 20-60 s with increments of 1, 2, and 5 s, respectively). After each light pulse, OIS was monitored for a t least 10 minutes for a CSD, If CSD occurred, no further pulses were delivered. If a baseline threshold was well determined, stimulations were started 3-4 steps before that baseline for subsequent CSDs. CSD triggered under isoflurane anesthesia was performed as described in Example 3 and Yousef Yengej, et al, J Physiol. 599 (2021): 4545-4559, which is hereby incorporated by reference in its entirety, particularly in relation to triggering of CSD.

To prevent possible bias during data analysis, data preparation and analysis was performed through fully automated procedures/algorithms using the same criteria, i.e., every data trace was analyzed in an identical manner. In this way, no blinding was needed for the analysis. Software was written in Python 3 and data was analyzed using Python, SciPy, Dask, Holoviews, and Igor.

Mice in either the sleep or the wake state were measured. Room lights were on a 12 hour cycle (off/on at 1 AM/1 PM, respectively). CSD was triggered in the awake state between 8 and 10 AM, and in the sleep state between 5 and 7 PM. Sleep states were determined by the head movement and verified with video recording. Angular momentum was filtered with a 5 min width running median filter. If filtered angular momentum was one standard deviation below average (of all angular momentum), the animal was considered to be asleep; otherwise, the animal was considered to be awake. To classify CSD as occurring during sleep, the animal must have been sleeping for at least 30 minutes before the CSD and during the CSD event, apparat from the transient awakenings (<2 min) that occur with 1 min of the CSD onset or completion. The few incidences where the animal work up for sustained duration (>2 min) during a “sleep CSD” or fell asleep during an “awake CSD” were excluded from analysis of the kinetics and amplitude of the CBV responses, to avoid the potentially confounding effect of a sustained switch in the sleep-wake state. Time asleep or awake was determined from the most recent change in state to the start of the CSD.

All comparisons were made from measurements from 6 animals, where a total for 47 CSDs (21 awake and 26 asleep) spread over the animals (for each animal n=3/3/8/8/11/14). To make comparisons for CSD properties such as amplitudes, durations, and heart rates in either the sleep or awake condition, we only considered CSDs where the animal was in the condition for a minimum of 10 minutes before the CSD and throughout the CSD. For each animal, a single “pure” CSD for each condition was measured (12 recordings, i.e. paired n=6). For the heart rate (HR) analysis, data from animal was excluded as the OIS signal insufficient to render a reliable HR (HR is paired n=5). To compare stimulation thresholds, average stimulation durations were calculated for each animal from all CSDs and these averages were compared (n=6). To determine correlation between the sleep or awake duration and the CSD threshold and amplitudes, all 47 CSDs from the 6 animals were used with a weighted Pearson correlation to correct for any possible nesting bias. For correlations between HR and movement, each animal contributed an equal number of points from the “pure” CSDs (from which there was one awake and one asleep “pure” CSD measured for each animal), and a Spearman correlation was used.

All values are reported as average±standard deviation. All hypothesis testing was two-tailed. Distributions were evaluated using histograms and kernel density estimate plots. For multiple comparison of single groups, paired Student's t-tests were used. For multiple comparison within each state, variability was examined between phases using one-way ANOVA (i.e., within the awake or asleep state). Tukey's HSD test was then used to find the differences between specific group means within each pool. Comparison between states was done per phase with a paired Student's t-test and resulting p-values were corrected using Holm=Bonferroni correction. Statistical significance was considered at p<0.05.

Different Thresholds for CSD in Different Brain States

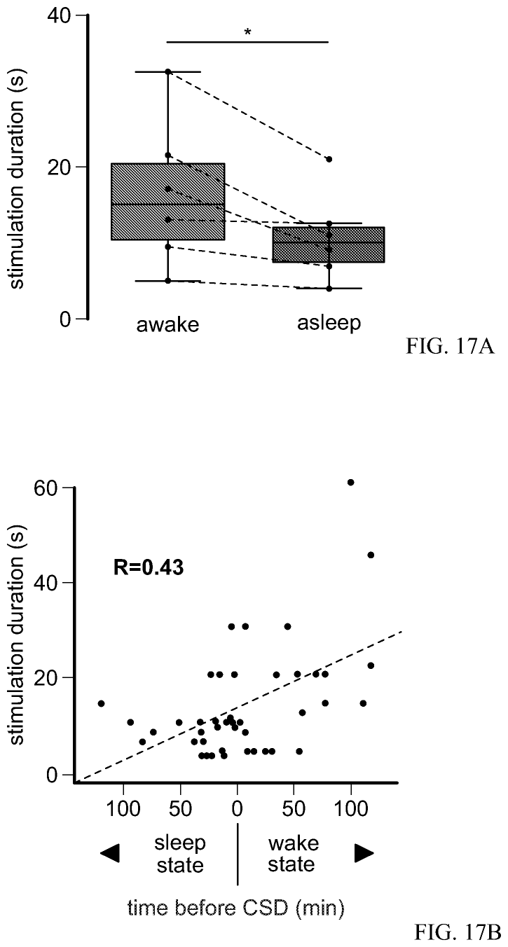

The microchip-system enabled intermittent optical triggering and recording of CSD in the same mouse over extended time periods, in different brain states. These measurements illustrate that threshold for optically triggering CSD can be quantified based upon the duration of light stimulation required to evoke CSD. The threshold for evoking CSD varied depending on the sleep-wake state of the mouse, but was consistent over multiple weeks for either state. The average threshold for triggering CSD was significantly higher in the wake state compared with the sleep state (stimulation duration=16.4±9.7 sec vs. 10.8±5.8 sec, resp., n=6, p=0.037) (FIG. 17A). Additionally, for the first 120 minutes in each state, there was a moderate correlation between the time in the sleep to wake states and the CSD threshold (R=0.43, FIG. 17B). When mice were under isoflurane anesthesia, the threshold for triggering CSD was also significantly lower than in the wake state, but not different from that observed in the sleep state (data not shown).

Neurovascular Characteristics of CSD in Different Brain States

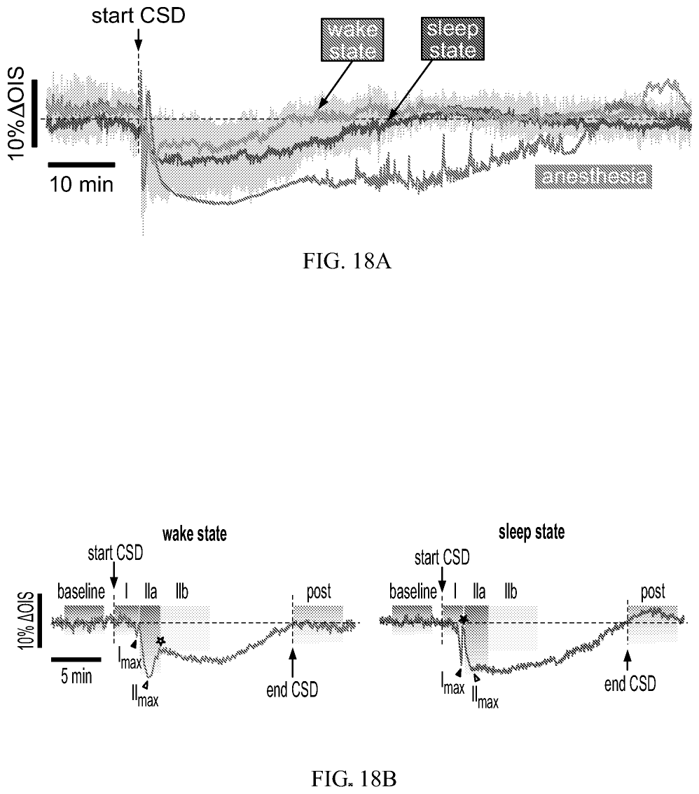

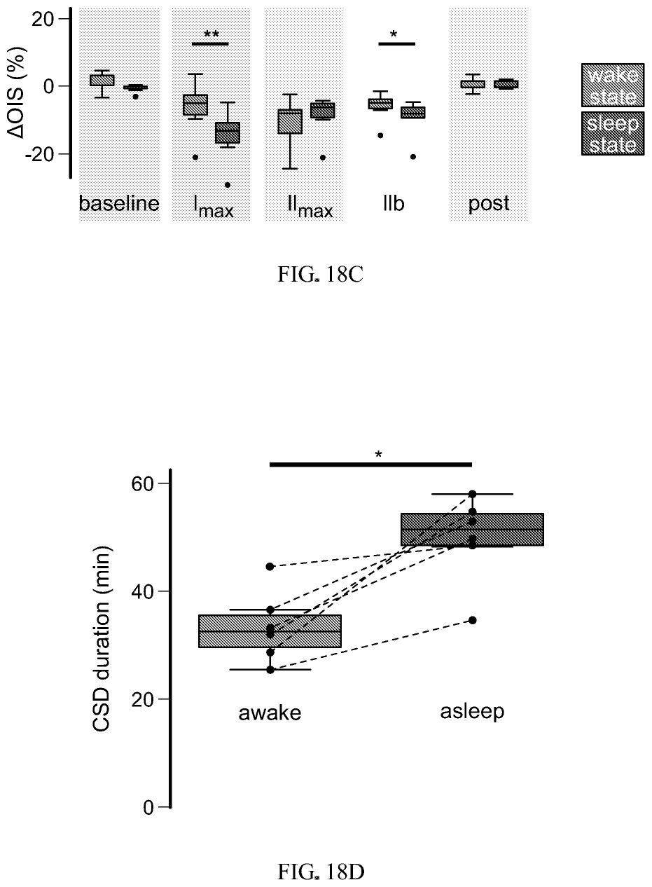

In the present studies, OIS primarily indicates CBV, which corresponds with constriction and dilation of cortical surface arteries. Under conditions of normal neurovascular coupling, increases and decreases in CBV are correlated with increases and decreases in neuronal activity. The microchip-based recording system showed changes in the OIS associated with CSD (FIGS. 18A-B) with similar characteristics to those observed with video recording. CSD in mice is associated with a complex multi-phasic neurovascular response, that includes an initial slowly propagated wave of vasoconstriction followed by a sustained global decrease in CBV.

The neurovascular response to CSD was divided into 5 different phases for the purposes of analysis (FIG. 18B). These are:
a baseline before CSD (quantified by Iₘₐₓ),
Phase I, the propagated CSD wave (seen on video recordings) lasting 3-5 minutes, which appears as a decrease in CBV followed by a recovery;
Phases IIa and IIb, sustained global decrease in CBV (30-60 min) with a short initial recovery, clearly observed in the awake state (IIa, quantified by IIₘₐₓ); and post CSD.

Different Neurovascular Characteristics of CSD in Different Brain States

Amplitude, duration, and other kinetics of the CBV response were significantly different in CSD in the wake vs. sleep state (n=6 animals). In the wake state, phase I of the CBV response was less distinct and significantly smaller in amplitude compared to the sleep state (Iₘₐₓ=−4.5±5.0% ΔOIS vs. −14.3±8.5% ΔOIS, p=0.001; FIG. 18A-C). The maximum decrease in CBV at phase IIa (IIₘₐₓ) was similar in the awake and sleep states (IIₘₐₓ=−10.81±7.6 vs. −9.2±6.4% ΔOIS, resp., p=0.486), but in phase lib the OIS was significantly different between wake and sleep (IIb −5.9±4.5 vs 9.4±5.4% ΔOIS, resp, p=0.037), due to a slight recovery in 1-2 minutes under the wake condition (FIG. 18A-C). The overall duration of the CSD, as measured by OIS, was significantly less in the wake state vs. the sleep state (33:22±6:37 vs. 49:42±8:05 min p=0.012; FIG. 18D).

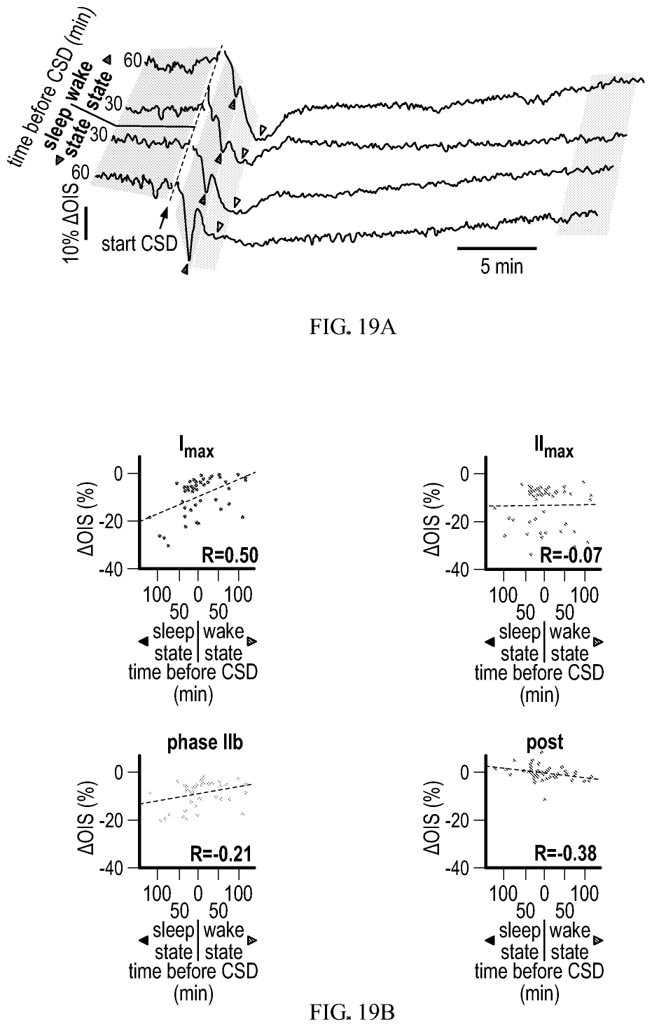

The kinetics of the responses also varied depending on the time in the wake vs. sleep states at which CSD was triggered. The characteristic changes in Iₘₐₓ, Phase IIb and post weakly to moderately correlate with the time in the sleep vs wake states (R=0.50, 0.21, −0.38 resp), becoming more apparent with prolonged duration in each state (FIGS. 19A and B).

Behavioral Responses to CSD in Different States

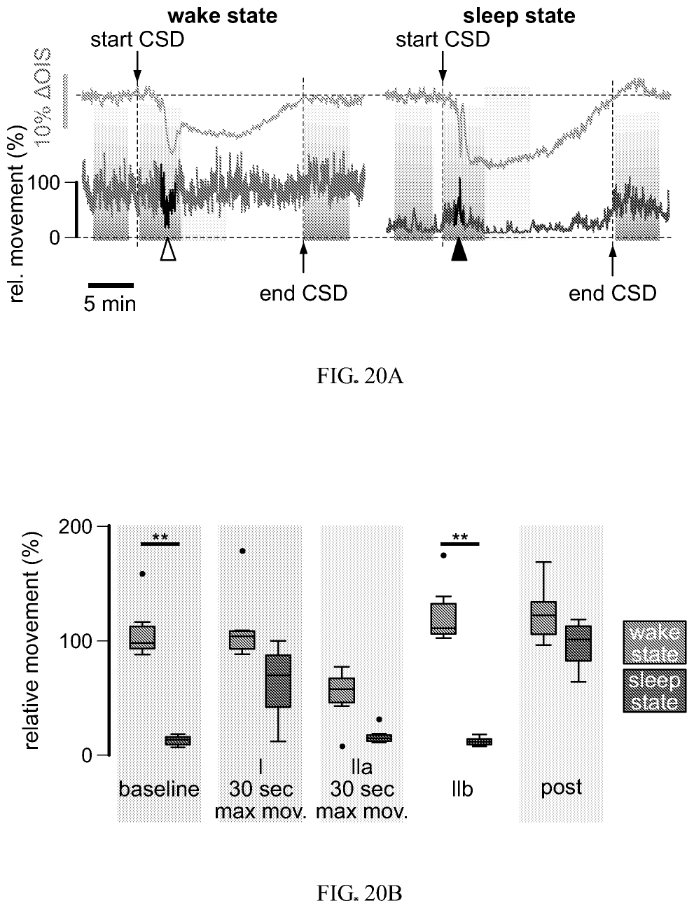

With CSD triggered in the wake state, mice showed a transient freezing behavior indicated by the movement sensor (FIGS. 20A and B) and verified by video recording. This corresponded with Phase IIa of the CBV response (109±96 vs. 52±25% relative to avg. awake movement, p=0.010), and lasted 1-2 minutes, after which there was a return to baseline movement. With CSD triggered during sleep, there was typically a transient awakening with increased movement (109±96 vs. 124±28%, p=0.819), in some cases accompanied by a characteristic behavior of head rubbing with the paws, and, in a few cases, a characteristic elongation of the body. This transient awakening and behavioral response corresponded with Phase I of CSD (12±4 vs. 62±34%, p=0.001), and lasted 1-5 minutes (FIGS. 20A and B). Following these behavioral responses, the mouse then typically returned to sleep approximately until the recovery of CBV to baseline levels, at which point the mouse awakened and showed significantly increased motor activity (12±4 vs. 96±22%, p=0.001), including head rubbing behavior similar to, but higher than that observed at the onset of CSD (p=0.033).

HR Response to CSD in Different States

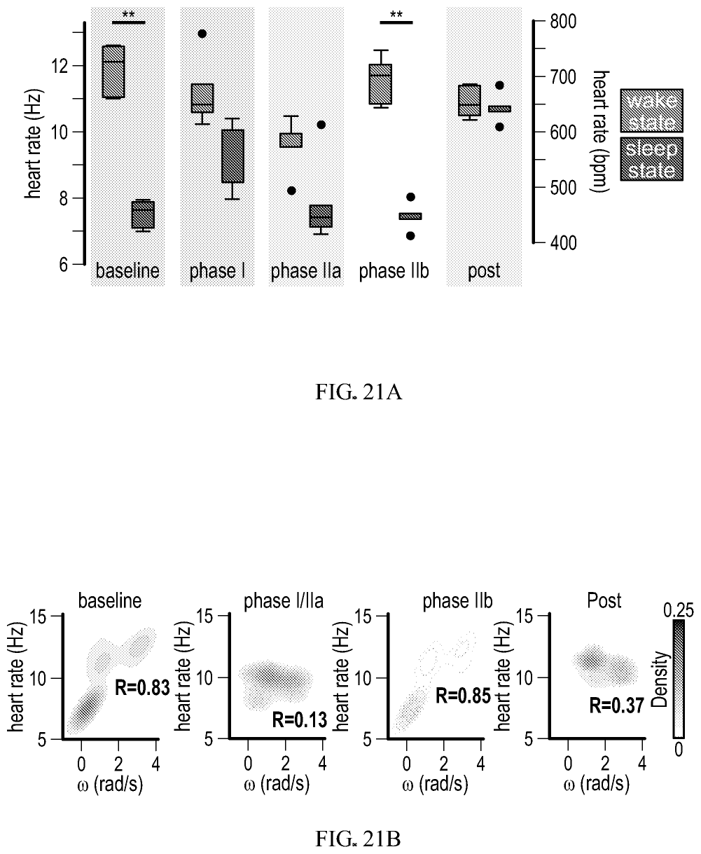

For the heart rate (HR) analysis, the data for one mouse was left out because the OIS signal was too noisy to render a reliably HR (therefore all HR data is n=5). Average HR was higher in the wake state than in the sleep state (11.9±0.8 Hz vs. 7.5±0.4 Hz, resp., p=0.001). When CSD was triggered in the wake state, there was a transient significant decrease in HR that corresponded with phase IIa of the CSD (FIG. 21A, baseline=11.9±0.8 Hz vs, IIa=9.6±0.8 Hz, p=0.002). Conversely, CSD triggered during sleep was associated with a transient increase in HR at phase I (FIG. 5A, baseline=7.5±0.4 Hz vs., 1=9.3±1.1 Hz, p=0.016). Comparison of the distribution of HR and movement for each phase of CSD showed that during the baseline preceding CSD and during Phase IIb, there was a strong correlation between HR and movement (R=0.83 during baseline, 0.85 during phase II). However, this correlation was not present during phase I/IIa (R=−0.13). Furthermore, at the end of the CSD CBV response, there was an inverse correlation (R=−0.37). These results indicate that at the onset and end of CSD, there is a disruption of the normal correlation between HR and movement (FIG. 21B).

These results illustrate that optogenetic approaches to triggering CSD are significantly less invasive than approaches in which CSD is triggered with KCl, electrical stimuli, or pinprick. Combining the optical triggering of CSD with minimally invasive recording approaches enables a refined understanding of the physiological and behavioral responses to CSD₉. Previous applications of this approach, however, require breaching the skull, and in some cases triggering CSD under anesthesia. The microchip-based approach described herein avoids these issues, enabling continuous synchronized high-resolution recording of multiple physiological and behavioral parameters and intermittent triggering of CSD over extended time periods without anesthesia. With this approach, CSD can be triggered repetitively in the same animal, to compare characteristics in different brain states or under different conditions. Responses to repetitive intermittent CSD over multiple weeks can be evaluated as a model of frequent or chronic migraine. The behavioral response to CSD can be more accurately characterized because of the absence of anesthesia or restrictions of movement, as well as the ability to perform studies without handling the mice, thus reducing stress that could further confound interpretation of behaviors. As an approach to therapy discovery and characterization, the micro-chip system can be used to evaluate CSD before, during, and after administration of therapies, thereby enhancing its utility as a translational model.

The multiphasic neurovascular changes associated with CSD may be relevant to the complex changes in cerebral blood flow (CBF) observed in migraine. The initial phases of triggered or spontaneous migraine attacks have in some cases been reported to be associated with a transient increase in CBF but are predominantly associated with cerebral hypoperfusion. In mice, the propagated CSD wave may be associated with a brief initial dilation of cortical surface arteries, but the predominant response to CSD is a profound decrease in CBV and vasoconstriction that occurs in multiple phases. The vascular changes recorded with our microchip-system may represent a model for those observed in migraine, although significant caution is warranted in the extrapolation of mouse studies to human migraine.

These results show significant differences in the characteristics of CSD that are triggered in different states. The wake state is associated with generally increased cortical activity, which might be expected to be associated with increased cortical excitability and therefore a reduced threshold for CSD and greater amplitude and duration of CSD. However, this data illustrates the opposite result, i.e. the threshold for evoking CSD was significantly lower in mice that were asleep or under anesthesia as compared with the wake state. Similarly, the amplitude and duration of the neurovascular response to CSD were larger during sleep or under anesthesiathan in the awake state. The specific mechanisms underlying these results are not yet clear, but similar mechanisms could also be involved in the increased occurrence of some types of seizures during sleep. Without wishing to be bound by theory, one possibility is that neurochemical fluctuations that occur during the circadian cycle could play a role in the characteristics of CSD in different brain states. This hypothesis is supported by the graded characteristics of the CSD response based upon the time since falling asleep or awakening. One possible chemical mediator is adenosine, which is believed to accumulate during wakefulness as a product of energy metabolism, contributing to the “metabolic drive” to sleep. Alterations in adenosine levels could influence the propensity for CSD and modulate its neurovascular characteristics. Multiple other physiological consequences of fluctuations in brain metabolism that occur during the sleep-wake cycle could also play a role. Alterations in the activity or synchronization of inhibitory vs. excitatory networks could also contribute to differences in the propensity for CSD and its characteristics in different states. Inhibitory intemeurons may be differentially active in the wake vs. sleep states. Increased activity of inhibitory interneurons has been reported to be involved in initiation of CSD.

CSD triggered in the wake state was associated with a freezing behavior, similar to those in rats. This transient freezing behavior has been reported to be inhibited by a CGRP receptor antagonist, raising the possibility that it could be related to migraine pain. It may also reflect a transient anxiety or fear response. Both in mice and rats, CSD was previously reported to cause a sustained reduction in motor activity, but this was not observed with CSD triggered optically in the wake state. These different results could be due to methodological differences, including an optical vs. KCl trigger, and whether CSD was evoked under anesthesia. CSD triggered during sleep was associated with transient awakening and a stereotypical behavior (head rubbing with the paws and sometimes transient body elongation, after which the mouse typically returned to sleep). Awakening and increased motor activity were consistently observed at the end of the sustained vasoconstriction (at the end of CSD). This observation suggests that although the propagated CSD wave did not produce sufficient symptoms to cause sustained awakening, a delayed behavioral response could be caused by the prolonged neurovascular changes associated with CSD. Consistent with this hypothesis, Zhang et al. J Nerurosci 30 (2010): 8807-8814, incorporated by reference in its entirety, reported that in rats under anesthesia sustained firing of trigeminal neurons commonly occurred with a delay of 20-30 minutes after the initial CSD wave. In rats, CSD has been reported to activate the thalamic reticular nucleus; this could represent a mechanism underlying the awakening occurring when CSD is triggered during sleep. CSD triggered optically in mice under anesthesia may produce sustained mechanical allodynia, changes in grimace scale, and behavior consistent with anxiety after recovery from anesthesia. These findings indicate a behavioral response to CSD that may include sustained discomfort and anxiety. The microchip system of the present disclosure will facilitate these assays in the future.

CSD triggered during the wake state caused a transient decrease in HR, while CSD triggered during sleep caused a transient increase in HR. Possibly changes in movement (freezing in the awake state vs. awakening and increased movement with sleep-triggered CSD) contributed to these different HR responses. However, the correlation between HR and movement observed at baseline in the sleep and wake states was disrupted by CSD, suggesting that other mechanisms contribute to the observed changes in HR. It is possible that both the movement and HR responses could be components of an orienting response provoked by CSD. CSD could also directly or indirectly activate autonomic responses, possibly due to changes in intercranial pressure such as the Cushing reflex, or due to anxiety and discomfort.

Migraine has a bi-directional relationship with the sleep-wake cycle. It is common for patients with migraine to awaken during the night with headache, or to have headache upon awakening in the morning. Migraine that occurs upon awakening is generally considered to be more severe and difficult to treat. Too little or too much sleep may be a migraine trigger, and conversely migraine attacks may disrupt normal sleep patterns. Migraine attacks that occur in the wake state may be associated with an intense urge to sleep and may be relieved by sleep. Migraine has been associated with advanced sleep phases in families expressing mutations in the gene encoding casein kinase 1 delta (CK1δ), and population studies also suggest shared genetic mechanisms between migraine and sleep disorders.

A lower threshold for triggering CSD during sleep may indicate that the sleep state increases the susceptibility to migraine mechanisms. It is commonly assumed that the increased severity and reduced medication responsiveness of migraine that occurs upon awakening is due to lack of awareness of early symptoms that delay acute treatment. Another possibility, however, is that migraine mechanisms that occur during sleep have more pronounced neurovascular effects that result in more severe and difficult-to-treat symptoms.

The microchip based systems of the present disclosure afforded the observation that CSD triggered during sleep resulted in only transient awakening may be consistent with the concept that aura symptoms associated with an initial CSD wave may not be sufficient to produce sustained awakening in those with migraine. By contrast, the awakening and behavior observed at the end of the sustained phase of vasoconstriction associated with CSD could be consistent with a delayed awakening that occurs once the pain phase of migraine is well established. The HR responses to CSD raise the possibility that “signatures” of CSD or other early migraine mechanisms could be identified using non-invasive monitoring with wearable technologies, leading to earlier and more effective therapy.

CSD occurs in the setting of brain injury and may influence clinical outcomes. It is common for brain-injured patients to be placed under sedation or general anesthesia. These results raise the possibility that sedation or general anesthesia could increase the propensity for CSD and amplify its neurovascular effects. It may therefore be worth considering the effects of different sedatives or anesthetics on CSD in the clinical management of brain-injured patients.

Studies are underway with higher resolution video recording and deep-learning assisted analysis in conjunction with microchip recording to enable improved quantification and analysis of behaviors. The studies were also not designed to evaluate specific stages of sleep; this is also a limitation that is being addressed in ongoing studies that include EEG as a simultaneous recording modality.

Nevertheless, these findings underscore the important influence of the sleep-wake cycle on basic mechanisms of migraine. An increased understanding of the specific mechanisms underlying the bi-directional relationship between the sleep-wake cycle and migraine has the potential to identify new therapeutic approaches not only for migraine, studied with the systems and methods for the present disclosure, but also for brain injury, sleep disorders, seizure disorders, and other neurological diseases.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

All documents cited or referenced herein, and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

1. A microchip system for the detection of neurovascular activity and/or behavior in a subject; wherein said microchip system is attachable to the skull of said subject and said microchip system comprises:
a) a source of electromagnetic measurement radiation arranged on said microchip system such that when said microchip system is attached to said skull, at least a portion of said electromagnetic radiation from said source reflects off of neural tissue and/or triggers fluorescence from neural tissue comprising one or more fluorescent molecules underlying said skull; and
b) an optical sensor arranged on said microchip system such that when said microchip system is attached to said skull, said optical sensor is configured to sense a portion of said reflected electromagnetic radiation off of or fluorescence from neural tissue comprising one or more fluorescent molecules underlying said skull;
wherein changes in the reflected and/or fluoresced electromagnetic radiation correlate with said neurovascular activity; and
wherein said microchip system is configured to receive signal from said optical sensor and said microchip system is configured to transmit an electrical signal corresponding to at least one property of said electromagnetic radiation.

2. (canceled)

3. The microchip system according to claim 1, wherein the source of electromagnetic radiation comprises a light emitting diode.

4-9. (canceled)

10. The microchip system according to claim 1, wherein said optical sensor has a sample rate of less than 20 kHz.

11. The microchip system according to claim 1, wherein the optical sensor transmits measurements to a microcontroller or processor for said recording.

12. The microchip system according to claim 1, wherein said optical sensor measures intensity to detect said changes.

13. The microchip system according to claim 1, wherein said microchip system further comprises a source of electromagnetic stimulus radiation; wherein said electromagnetic stimulus radiation is electromagnetic radiation emitted from said source towards neural tissue underlying said skull; and
wherein said stimulus electromagnetic radiation induces a response in said subject.

14. The microchip system according to claim 13, wherein said subject is a subject expressing opsin, or a fluorescent molecule, or said subject neural tissue comprising a fluorescent molecule.

15. The microchip system according to claim 13, wherein said source of electromagnetic stimulus radiation comprises a light emitting diode.

16-18. (canceled)

19. The microchip system according to claim 1, wherein said microchip system further comprises a motion sensor and/or
a thermal sensor for measurement of skull surface temperature and/or
a pressure sensor for measurement of intracranial pressure and/or
a heat source.

20-22. (canceled)

23. The microchip system according to claim 1, wherein said microchip system comprises a wireless transmitter for transmission of measurements and/or a connector for a wired connection between said microchip system and said recording device.

24-26. (canceled)

27. The microchip system according to claim 1, wherein said microchip system has a weight of less than 10 g.

28-37. (canceled)

38. A system for the detection of neurovascular activity and/or behavior in a subject comprising:
a) one or more microchip systems according to claim 1,
b) a recording system in communication with the microchip system, wherein each microchip system transmits measurements from each sensor independently.

39-41. (canceled)

42. The system according to claim 38, wherein said system further comprises an enclosure for said subject.

43. The system according to claim 42, wherein said system further comprises a camera for monitoring the movement of said subject in said enclosure.

44. The system according to claim 38, wherein said system comprises a microcontroller and optionally a camera, said microchip system comprises a motion sensor, and
said microcontroller is configured to identify awake and sleep cycles of said subject based on measurements transmitted thereto from said motion sensor and/or said camera.

45. The system according to claim 44, wherein said enclosure is attached to a vibration system.

46. A method for the detection of neurovascular activity in a subject having a microchip system according to claim 1, attached to the skull of said subject, comprising:
a) emitting electromagnetic measurement radiation from said source of electromagnetic measurement radiation; and
b) measuring the reflected or fluoresced electromagnetic measurement radiation with said optical sensor;
wherein changes in the reflected or fluoresced electromagnetic radiation correlate with said neurovascular activity.

47-49. (canceled)

50. A method for the detection of neurovascular activity and behavior in one or more subjects comprising:
a) placing each of said one or more subjects into an enclosure in one of the systems according to claim 42; wherein each of said one or more subjects has said microchip system affixed to the skull of said subject;
b) emitting electromagnetic radiation from said sources of electromagnetic measurement radiation on said microchip system to reflect off of or fluoresce from neural tissue underlying the skull of each of said subject; and
c) measuring the reflected or fluoresced light in the optical sensor from each of said microchip systems;
wherein changes in the reflected electromagnetic radiation correlate with said neurovascular activity.

51. The method according to claim 50, wherein said emitting and measuring steps occur over more than 5 sleep-wake cycles of said subject.

52. A method of triggering and monitoring cortical spreading depression (CSD) in a subject expressing channelrhodopsin, wherein said subject expressing channelrhodopsin has a microchip system attached to the skull of said subject, and said microchip system comprises an optical microchip system having:
a) a source of electromagnetic measurement radiation arranged on said microchip system such that when said microchip system is attached to said skull, at least a portion of said electromagnetic radiation from said source reflects off of or fluoresce from neural tissue underlying the skull of each of said subject;
b) an optical sensor arranged on said microchip system such that when said microchip system is attached to said skull, said optical sensor is configured to senses a portion of said reflected electromagnetic radiation and/or fluorescence radiation; and
said microchip system comprises an electromagnetic stimulus microchip system comprising:
a) a source of electromagnetic stimulus radiation; wherein said electromagnetic stimulus radiation is electromagnetic radiation emitted from said source towards said skull; and
wherein said stimulus electromagnetic radiation induces CSD in said subject;
wherein changes in the reflected electromagnetic measurement radiation correlate with said neurovascular activity; and
wherein said microchip system is configured to receive signal from said optical sensor and said microchip system is configured to transmit an electrical signal corresponding to at least one property of said electromagnetic radiation;
said method comprising:
a) emitting electromagnetic stimulus radiation from said stimulus radiation to induce CSD in said subject;
b) emitting electromagnetic measurement radiation from said source of electromagnetic measurement radiation; and
c) measuring the reflected electromagnetic measurement radiation with said optical sensor.

53-60. (canceled)
